(19) United States
(12) Patent Application Publication
(10) Pub. No.: US 2024/0416145 A1
(43) Pub. Date: Dec. 19, 2024

(54) RADIOACTIVE SOURCE DELIVERY SYSTEM WITH A STYLET PULLING MECHANISM AND ITS METHOD OF USE

(71) Applicant: HANGZHOU DASHTECH CO., LTD, Hangzhou (CN)

(72) Inventors: Xuetang WANG, Hangzhou (CN); Dingchen Zhu, Hangzhou (CN); Guangming Fu, Hangzhou (CN); Xingxing Lei, Hangzhou (CN)

(21) Appl. No.: 18/821,881

(22) Filed: Aug. 30, 2024

Related U.S. Application Data
(63) Continuation of application No. PCT/CN2023/078879, filed on Feb. 28, 2023.

(30) Foreign Application Priority Data
Mar. 3, 2022 (CN) 202210207077.0
Mar. 3, 2022 (CN) 202210208833.1
Jul. 1, 2022 (CN) 202210773948.5
Aug. 26, 2022 (CN) 202211032981.9
Dec. 9, 2022 (CN) 202211586593.5
Jan. 5, 2023 (CN) 202310014707.7
Jan. 17, 2023 (CN) 202310063072.X

(51) Int. Cl. A61N 5/10

(52) U.S. Cl. CPC A61N 5/1007; A61N 2005/1009; A61N 2005/101

(57) ABSTRACT

The invention discloses a radioactive source delivery system with a stylet pulling mechanism and its method of use. The wire driving mechanism is connected to the wire output channel, and the wire driving mechanism is used to drive the pushing wire to move back and forth along the wire output channel. A stylet pulling mechanism is located on one side of the front end of the wire output channel. Through the channel-switching mechanism, the wire output channel is docked with a flexible delivery tube. The front end of the flexible the delivery tube is connected to a puncture needle or has a quick connector for connecting to the puncture needle. The stylet pulling mechanism can dock with the tail portion of the flexible stylet inside the flexible delivery tube, pulling the flexible stylet out of the flexible delivery tube to form a hollow flexible delivery channel. A radioactive source feeding mechanism is used to place the radioactive source in front of the pushing wire. The wire driving mechanism drives the pushing wire forward, pushing the radioactive source through the wire output channel, flexible delivery tube, and into the puncture needle inserted in the target object, ultimately implanting it into the target object. This invention prevents blood from entering and clotting inside the puncture needle, reduces surgery time, achieves full automation, and improves the accuracy of the implantation position.

This application is a continuation of International Patent Application No. PCT/CN2023/078879, filed on Feb. 28, 2023, the entire contents of which are hereby incorporated by reference.

### TECHNICAL FIELD

This invention belongs to the technical field of medical devices, specifically relating to a Radioactive Source delivery System with a stylet pulling mechanism and its method of use which is used in radioactive source implantation surgeries.

### BACKGROUND TECHNOLOGY

Radioisotope seeds implantation is a technique which refers to directly implanting isotopic radioactive sources into tumor areas for treatment. It belongs to one of radiation therapies. Currently, this technique implants radioactive sources into or around the tumor via puncture needle with guidance of imaging data provided by CT or ultrasound image. The radioactive sources continuously emit radiation to destroy the tumor cells. The implanted radioactive sources are typically iodine-125 seeds, with a half-life of 59.6 days and a radiation radius of less than 1.7 cm within the human body.

So the seeds have the advantage of being safe and shielding easily. They emit gamma rays which effectively irradiate tumor cells for a continuous period of 180 days. This technique has the characteristic of high-dose distribution within tumor areas to kill tumor cells while surrounding normal tissues receive minimal radiation, causing little to no damage. Essentially, this technique is a form of precise radiotherapy.

Chinese Pub. No. CN1069415A, CN1069063C, CN1190602A, CN1322578A, and CN2235827Y disclose a treatment method and device suitable for various tumors in the human body. Before treatment, a tube or a needle connected with a tube is inserted into the tumor site in the human body, and the radioactive source is fixed to the end of a steel wire rope. It is then delivered through the tube to the tumor site for therapy. After treatment, the steel wire rope and radioactive source are retrieved. In such brachytherapy, the tip of the tube or puncture needle is sealed (while in the seed implantation surgery, the tip of the hollow puncture needle is open for implanting seeds). And the puncture needle is connected to the front end of a flexible tube, and a radioactive source delivery device is installed at the rear end of the flexible tube. The radioactive source is delivered forward along the tube to the tumor (the radioactive source is not implanted into the body but emits radiation through the tube or the puncture needle). The radiation intensity of this radioactive source is much stronger than that of the I-125 seeds used in seed implantation surgery, achieving radiation therapy effects in just a few minutes. Compared to seed implantation surgery, this kind of therapy has a less irradiate time and cannot suppress tumor growth for an extended period. So, its efficacy in treating certain cancers is not as good as seed implantation surgery. Yet, the seed implantation surgery requires manual intervention to adjust the depth of seed implantation by pulling the needle trocar partly out of the body, which leads to the issue of doctors being exposed to radiation. Additionally, in the seed implantation surgery, the radioactive seeds need to be implanted into the body, so the implantation system need to overcome disinfection and biocompatibility challenges.

Chinese Pub. No. CN110496301A, CN 211214946U, WO2021022971A1 have disclosed a robot for targeted seed implantation in the lithotomy position suitable for clinical use. The robot comprises a frame, a pose adjustment mechanism, a friction wheel seed implantation device with tactile feedback, and a sine elastic amplification torque compensation mechanism. The use of a sine elastic amplification torque compensation mechanism allows for the compensation of gravitational torque in any arbitrary position of the arm, reducing the fluctuation of driving torque and improving the stability of the robot's low-speed operation at the end effector. However, the robot is rigidly connected to the puncture needle, which not only easily scratches the patient during the surgical process, posing a danger, but also leads to a complex and bulky connection structure at the tail end of the puncture needle. It limits the implantation location of radioisotope seeds, and increases the difficulty and duration of the surgical operation.

To address this issue, one solution is to connect a flexible tube between the seed implantation device and the puncture needle, delivering the seed through the flexible tube to avoid rigid connection. However, there is a stylet inside the puncture needle that cannot be removed before the implantation to prevent blood from entering and clotting inside the puncture needle, causing a blockage. So that an automatic stylet pulling and storage mechanism is needed. This mechanism should be able to pull out the stylet before implantation and then immediately proceed with the implantation.

### SUMMARY OF THE INVENTION

To solve the above technical problems, the purpose of this invention is to provide a radioactive source delivery system with a stylet pulling mechanism and its method of use. The stylet pulling mechanism can dock with the tail portion of the stylet inside the flexible delivery tube and pull the stylet out of the flexible delivery tube, forming a hollow implantation channel. This facilitates the wire driving mechanism to drive the pushing wire to push the seed or seed strand along the flexible delivery tube to the target position.

To achieve the aforementioned objectives, this invention adopts the following technical solutions:

A radioactive source delivery system comprises a radioactive source delivery mechanism, a stylet pulling mechanism. The radioactive source delivery mechanism includes a wire output channel, a pushing wire, and a wire driving mechanism. The wire driving mechanism connects to the wire output channel, which is used to drive the pushing wire to move back and forth along the wire output channel. The wire output channel is a rigid structure or a flexible structure that can be bent. The stylet pulling mechanism arranged in parallel with the front end of the wire output channel. The stylet pulling mechanism includes a friction stylet pulling component. A part of the friction stylet pulling component presses tightly against the stylet, so the needle stylet is pulled by the frictional force generated by the pressing.

Preferably, the friction stylet pulling component is one or a combination of a friction wheel component, a friction belt component, or a reciprocating clamping component. The friction wheel component or friction belt component includes one or more sets of friction wheels or friction belts. One side of which can be in close contact with the stylet. The rotation of the friction wheels or the cyclical movement of the friction belts drives the stylet to be pulled out. The reciprocating clamping component comprises a reciprocating motion component and a clamping component. The clamping component is mounted on the reciprocating motion component and can move back and forth along a certain trajectory driven by the reciprocating motion component. The clamping component can clamp the stylet when driven in the direction of the stylet pulling by the reciprocating motion component, pulling out the stylet. Conversely, when driven in the opposite direction by the reciprocating motion component, the clamping component releases the stylet, then resetting.

Preferably, it further comprises the flexible delivery tube with a flexible stylet inside. The front end of the flexible stylet extends from the flexible delivery tube, and fills the space inside the trocar needle connected with the front end of the flexible delivery tube, which prevents blood from flowing into the trocar needle, otherwise blood coagulation can lead to the blockage of the trocar needle. The stylet tail portion of the flexible stylet extends from the rear end of the flexible delivery tube and the stylet is equipped with a stop step. The stop step is in contact with one end of the flexible delivery tube, serving as a positioning function to prevent the front end of the stylet from protruding out of the trocar needle and piercing into the target body.

Preferably, the stylet pulling component docks with the stylet tail portion inside one of the flexible delivery tubes through a channel-switching mechanism, and to pull out the stylet, removing the stylet from the flexible delivery tube, thereby forming a hollow flexible delivery channel. The flexible delivery tube is the first flexible delivery tube. The stylet is a flexible wire with elasticity, which can be bent under the external force and can be restored to a straight state after the external force is removed. The material of the stylet is one or a combination of several types, including Ni—Ti alloy, spring steel, and composite materials. The length of the stylet is greater than 600 mm.

Preferably, the pushing wire is a flexible pushing wire, and the wire driving mechanism is a flexible wire driving mechanism. The flexible pushing wire is a flexible wire with elasticity, which can be bent under the external force and can be restored to a straight state after the external force is removed. The material of the flexible pushing wire is one or a combination of several types, including Ni—Ti alloy, spring steel, and composite materials. The length of the flexible pushing wire is greater than 600 mm.

Preferably, the wire output channel docks to one of the flexible delivery tubes through a channel-switching mechanism. The front end of the flexible delivery tube is connected with a trocar needle or has a quick connector designed to connect with a trocar needle. The quick connector is fixedly connected to the trocar needle using one or a combination of thread, latch, and adhesive. The radioactive source delivery mechanism also includes a radioactive source feeding mechanism, which is used to set a radioactive source at the front of the pushing wire. The wire driving mechanism drives the pushing wire forward to push the radioactive source along the wire output channel, through the flexible delivery tube, and into the target body via the trocar needle that has been inserted into the target body.

Preferably, the radioactive source feeding mechanism is a cutting mechanism. In this case, the pushing wire itself is a seed strand or a seed strand casing, or the front half of the pushing wire is a seed strand or seed strand casing, and the rear half of the pushing wire is a pushing wire. The seed strand and seed strand casing can be cut by the cutting mechanism, and the target length of the seed strand or seed strand casing is severed from the front end of the pushing wire by the cutting mechanism, thereby achieving the feeding of the seed strand or seed strand casing. When the severed part is a seed strand casing, the radioactive source feeding mechanism also includes a seed embedding mechanism. This seed embedding mechanism is capable of inserting seeds and/or spacers into the seed strand casing from one end or the side, to form a complete seed strand. The cutting mechanism is positioned at any location along the wire output channel.

Alternatively, the radioactive source feeding mechanism uses a magazine for feeding. The radioactive source feeding mechanism is directly integrated into the wire output channel. Seeds, or seed strands, or seed strand casings are loaded into the storage slot or hole within the magazine. The feeding mechanism installed on the magazine is used to place seeds, or seed strands, or seed strand casings at the front end of the pushing wire for feeding. When the magazine is loaded with a seed strand casing, the radioactive source feeding mechanism also includes a seed embedding mechanism. This seed embedding mechanism is capable of inserting seeds and/or spacers into the seed strand casing from one end or the side, forming a complete seed strand.

Alternatively, the radioactive source feeding mechanism uses a seed strand driving mechanism for feeding seed strands. The radioactive source feeding mechanism comprises a seed strand driving mechanism, a seed strand output channel, and a cutting mechanism. It outputs seed strands or seed strand casings by the seed strand driving mechanism and cuts them to the desired length by the cutting mechanism, to achieve the feeding of seed strands or seed strand casings. When the seed strand driving mechanism outputs a seed strand casing, the radioactive source feeding mechanism also includes a seed embedding mechanism. This seed embedding mechanism is capable of inserting seeds and/or spacers into the seed strand casing from one end or the side, thereby forming a complete seed strand. The seed strand driving mechanism is connected to the seed strand output channel. The seed strand output channel is a rigid structure or a flexible structure that can be bent. When the seed strand output channel is connected with the Y-shaped channel or the second motion platform, the severed seed strand is transported to the output channel and is located ahead of the pushing wire.

Preferably, the channel-switching mechanism includes a first motion platform and a first connecting portion. The first motion platform with one end of the wire output channel and the first connecting portion respectively set on the opposite sides. The first motion platform is one of the following modes:
A. The first connecting portion moves and one end of the wire output channel remains stationary;
B. The first connecting portion remains stationary and one end of the wire output channel moves;
C. The first connecting portion moves and one end of the wire output channel also moves.

The first motion platform includes a planar motion mechanism and a first front-and-back docking mechanism. The first front-and-back docking mechanism connects to the planar motion mechanism. The planar motion mechanism is used to drive the first front-and-back docking mechanism to move within a plane. The first front-and-back docking mechanism drives one end of the wire output channel or the first connecting portion to move back and forth in a direction perpendicular to that plane.

The planar motion mechanism takes rotational movement in one direction to achieve the motion of the first front-and-back docking mechanism in a plane. Alternatively, the rotary motion in one direction and the linear motion at least in one direction of the planar motion mechanism drives the motion of the first front-and-back docking mechanism in a plane. Alternatively, the planar motion mechanism takes the linear motion in two directions to achieve two degrees of freedom motion of the first front-and-back docking mechanism in space.

Preferably, the first connecting portion connects to the connection part which has a plurality of connection holes. The one end of each flexible delivery tube is installed at the corresponding connection hole. The first motion platform is used to achieve the connection between one end of the wire output channel with any flexible delivery tube on the connection part, forming a delivery channel for radioactive source, achieving multi-channel delivery. The first connecting portion is one or a combination of an adhesive connection part, a welding connection part, a threaded connection part, a snap-fit connection part, or a locking connection part.

Preferably, it further comprises a second motion platform. The stylet tail portion protrudes from the connection hole of the connection part. The second platform with a stylet pulling mechanism and a second connection part respectively installed on opposite sides, which is used to achieve the relative motion in space of the stylet pulling component and the second connection part. It makes the stylet pulling component docks with the stylet tail portion inside each flexible delivery tube attached to the connection part, performing the pulling of the stylet, pulling it out from the flexible delivery tube, thereby achieving multi-channel stylet pulling.

The second motion platform is one of the following modes:
A. The second connection part moves while the stylet pulling component remains stationary;
B. The second connection part remains stationary while the stylet pulling component moves;
C. The second connection part moves while the stylet pulling component also moves.

Alternatively, the second motion platform is the first motion platform. In this scenario, the stylet pulling component and one end of the wire output channel are installed at the same side of the first motion platform. The second connection part is the first connection part. The first motion platform also includes a second front-and-back docking mechanism which connects to the planar motion mechanism. The first front-and-back docking mechanism and the second front-and-back docking mechanism are respectively used for the front-and-back docking motion of one end of the wire output channel and the stylet pulling component. First, the stylet pulling component docks with the stylet tail portion inside one of the flexible delivery tubes on the first connection part, and pulls the stylet. After the stylet pulling is completed, a new hollow implantation channel is established. Then, the first motion platform drives the wire output channel to dock with the flexible delivery tube, and then the implantation is carried out through the newly established implantation channel.

Preferably, it further comprises a stylet storage mechanism. The stylet storage mechanism is used to store the stylets pulled out by the stylet pulling component and located at the rear end of the stylet pulling component. When the stylet is output from the rear end of the stylet pulling component, the stylet storage mechanism stores the flexible stylet accordingly. Alternatively, the stylet storage mechanism is a part of the stylet pulling mechanism, completing the storage of the stylet while pulling the stylet. The stylet storage mechanism is a wheeled storage mechanism or a storage tube.

Preferably, the wheeled storage uses a coil wheel component. The coil wheel component includes a storage wheel and a storage wheel driver. The storage wheel driver drives the storage wheel to rotate, causing the stylet to be wound around the outer surface of the storage wheel.

Alternatively, the wheeled storage adopts a wheel-shaped stylet container which has a concave inner surface and has an opening on the side. The stylet extends into the concave inner surface through the side opening. The wheel-shaped stylet container is designed to rotate freely or be actively driven, positioned behind the stylet pulling mechanism. Under the action of the stylet's own elasticity, the stylet is automatically wound within the concave inner surface of the wheel-shaped stylet container.

Preferably, the stylet storage tube can be any one of a straight tube, a spiral tube, or a thin-film tube. The material of the stylet storage tube is one or a combination of metal, plastic, rubber, latex, silicone, or elastomer material. The storage tube contains a lubricant to facilitate the smooth insertion of the stylet, or the inner surface of the storage tube can be evenly coated with grease or the inner surface of the storage tube can take a lubricating coating to achieve the lubricating effect, and the material of the lubricating coating is Teflon. The inlet end of the storage tube has an elastic extension section, which can shorten under the action of press and automatically extend to its original position after the press is released.

Preferably, it further comprises a stylet insertion mechanism. The stylet insertion mechanism can insert the stylet along the flexible delivery tube into the trocar needle, filling the space inside the trocar needle, which prevents blood from flowing into the trocar needle, otherwise blood coagulation can lead to the blockage of the trocar needle.

Alternatively, the stylet pulling component is also the stylet insertion mechanism, which adopts a friction stylet pulling component. A part of this component presses tightly against the stylet. The friction stylet pulling component is capable of being driven to rotate in both directions, and it uses the frictional force generated by pressing to achieve the extraction and insertion of the stylet.

Preferably, the stylet tail portion is equipped with a stop step. The stylet insertion mechanism achieves precise positioning of the stylet through limiting action of the stop step and the rear end face of the flexible delivery tube. This ensures that the front end of the stylet reaches the front end of the trocar needle without protruding further forward.

Alternatively, the stylet insertion mechanism is equipped with a displacement measurement device. This device can measure the actual displacement of the stylet. When the displacement measurement device detects that the front end of the stylet reaches the front end of the trocar needle, the stylet insertion mechanism ceases its operation.

Preferably, it further comprises a third motion platform. The third motion platform with the stylet insertion mechanism and the third connection part respectively installed on the opposite sides, which is used to achieve the relative motion in space of the stylet insertion mechanism and the third connection part. It makes the stylet insertion mechanism docks with each flexible delivery tube on the third connection part and insert the stylet through that flexible delivery tube into the trocar needle, achieving multi-channel stylet insertion.

Alternatively, it makes the stylet insertion mechanism docks with the stylet disposal tube mounted on the third connection part and insert the pulled flexible stylet into the stylet disposal tube. The stylet disposal tube can be quickly assembled and disassembled with the first connection part to achieve the storage of the stylet.

The third motion platform is one of the following modes:
A. The third connection part moves and the stylet insertion mechanism remains stationary;
B. The third connection part remains stationary and the stylet insertion mechanism moves;
C. The third connection part moves and the stylet insertion mechanism also moves.

Alternatively, the third motion platform is the first motion platform. In this scenario, the stylet insertion mechanism and one end of the wire output channel are installed at the same side of the first motion platform. The third connection part is the first connection part. The first motion platform also includes a third front-and-back docking mechanism which connects to the planar motion mechanism.

The third front-and-back docking mechanism and the first front-and-back docking mechanism are respectively used for the back and forth docking motion of one end of the wire output channel and the stylet insertion mechanism. First, connecting the wire output channel with one of the flexible delivery tubes on the third connection part, and then proceeding with the implantation. After the implantation is completed, the stylet insertion mechanism docks with this flexible delivery tube and inserts the stylet through the flexible delivery tube into the trocar needle.

Preferably, the stylet pulling component is installed on the first motion platform through a quick connecting structure, which is one or a combination of a threaded connecting structure, a latch connecting structure, or a lock connecting structure.

The use method of a radioactive source delivery system with a stylet pulling mechanism includes the following steps:
a. Connecting the front ends of several flexible delivery tubes to the tail end of a trocar needle that has already been inserted into the target body. The stylet is inside the flexible delivery tube, the front end of the stylet extends to the trocar needle, so as to filling the space inside the trocar needle. A small section of the stylet extends out as a tail from the rear end of the flexible delivery tube.
b. Before implanting a radioactive source through one of the trocar needles, it is necessary to dock the stylet pulling component with the flexible delivery tube connected to this trocar needle. The stylet pulling component clamps to the stylet tail portion which extends from the rear end of the flexible delivery tube, so as to pull out the stylet from the flexible delivery tube and form a hollow delivery channel.
c. Docking one end of the wire output channel with this flexible delivery tube. The radioactive source delivery mechanism then pushes the radioactive source forward along the wire output channel, the flexible delivery tube, and the trocar needle, until it is implanted within the target body.

Preferably, step c is implemented by the docking motion of the first motion platform. Initially, one end of the flexible delivery tube is mounted on the first connection part. One end of the wire output channel and the first connection part are respectively installed at the opposite sides of the first motion platform. The first motion platform is used to realize the relative motion in space between the wire output channel and the first connection part. This allows the wire output channel connects to each flexible delivery tube on the first connection part, forming a delivery channel for radioactive source, so that the multi-channel implantation is achieved. The first motion platform is one of the following modes:
A. The first connection part moves and the wire output channel remains stationary;
B. The first connection part remains stationary and the wire output channel moves;
C. The first connection part moves and the wire output channel also moves.

Preferably, step b is implemented by the docking motion of the first motion platform. Initially, one end of a plurality of flexible delivery tubes are mounted on the first connection part. The stylet pulling component and the first connection part are respectively installed at the opposite sides of the first motion platform. By the first motion platform, relative motion in space is achieved between the stylet pulling component and the first connection part. This enables the stylet pulling component to dock with the stylet tail portion of the stylet inside each flexible delivery tube on the first connection part, and to pull out the stylet, then the stylet is pulled out from the flexible delivery tube, accomplishing multi-channel stylet pulling out. The first motion platform is one of the following modes:
A. The first connection part moves and the the stylet pulling component remains stationary;
B. The first connection part remains stationary and the the stylet pulling component moves;
C. The first connection part moves and the the stylet pulling component also moves.

First, docking the stylet pulling component with the stylet tail portion of the stylet inside one of the flexible delivery tubes on the first connection part, and pulling the stylet. After the stylet pulling is completed, a new implantation channel is established. Docking the wire output channel with the flexible delivery tube, and then the implantation is carried out.

### Beneficial Effects

Compared with existing technology, this invention features an automatic stylet pulling mechanism and a stylet storage mechanism. The stylet inside the puncture needle is only pulled before implantation, minimizing blood coagulation within the puncture needle tube, which can lead to puncture needle blockages. The stylet pulling mechanism allows for repeated stylet pulling and stores the stylet in the storage mechanism, addressing the issue of the stylet getting stuck within the stylet pulling mechanism when multiple stylets are pulled successively.

This invention enables multi-channel implantation by incorporating a first motion platform and a first connection part. The first connection part has multiple connection holes, with one end of each flexible delivery tube mounted in the corresponding connection hole. One end of the wire output channel and the first connection part are installed at opposite ends of the first motion platform. The first motion platform facilitates relative movement between the front end of the wire output channel and the first connection part in space, allowing the wire output channel to dock with each flexible delivery tube on the first connection part to form a delivery channel for the radioactive source, thus achieving multi-channel implantation. The structure is simple and reasonable, and the operation is convenient and quick.

This invention supports multi-channel stylet pulling by incorporating a second motion platform. One end of multiple flexible delivery tubes is mounted on the first connection part, with the tail end of the stylet exposed from the connection hole of the first connection part. The stylet pulling mechanism and the first connection part are mounted at opposite ends of the second motion platform.

The second motion platform enables relative movement between the stylet pulling mechanism and the first connection part in space, allowing the stylet pulling mechanism to dock with the tail end of the stylet within each flexible delivery tube on the first connection part, and pull the stylet from the flexible delivery tube, thus achieving multi-channel stylet pulling. Alternatively, the second motion platform can be the first motion platform, further simplify the structure by using the first motion platform to achieve relative movement between the stylet pulling mechanism and the first connection part in space.

### DESCRIPTION OF THE DRAWINGS

The schematic embodiments and descriptions of this application are intended to illustrate the invention and do not constitute limitations on its scope.

FIG. 1: Schematic diagram of the stylet pulling mechanism of Embodiment 1 (without stylet storage mechanism);

FIG. 2: Overall structural diagram of the friction stylet pulling mechanism of Embodiment 1;

FIG. 3: Internal structural diagram of the friction stylet pulling mechanism of Embodiment 1;

FIG. 4: Rear structural diagram of the friction stylet pulling mechanism of Embodiment 1;

FIG. 5: Schematic diagram of the stylet pulling mechanism of Embodiment 1 (with storage tube);

FIG. 6: Cross-sectional view at the position of the friction stylet pulling mechanism and the storage tube in FIG. 5;

FIG. 7: First schematic diagram of the working principle of the stylet pulling mechanism of Embodiment 1;

FIG. 8: Second schematic diagram of the working principle of the stylet pulling mechanism of Embodiment 1;

FIG. 9: Structural diagram of the stylet pulling mechanism of Embodiment 2;

FIG. 10: First internal structural diagram of the stylet pulling mechanism of Embodiment 2;

FIG. 11: Structural diagram of the friction wheel assembly of the stylet pulling mechanism of Embodiment 2;

FIG. 12: Structural diagram of the friction wheel pressing mechanism of the stylet pulling mechanism of Embodiment 2;

FIG. 13: Second internal structural diagram of the stylet pulling mechanism of Embodiment 2;

FIG. 14: Third internal structural diagram of the stylet pulling mechanism of Embodiment 2;

FIG. 15: Structural diagram of the measuring wheel and braking mechanism of the stylet pulling mechanism of Embodiment 2;

FIG. 16: First structural diagram of the laser detection module of the stylet pulling mechanism of Embodiment 2;

FIG. 17: Second structural diagram of the laser detection module of the stylet pulling mechanism of Embodiment 2;

FIG. 18: Third structural diagram of the laser detection module of the stylet pulling mechanism of Embodiment 2;

FIG. 19: First structural diagram of the camera detection module of the stylet pulling mechanism of Embodiment 2;

FIG. 20: Second structural diagram of the camera detection module of the stylet pulling mechanism of Embodiment 2;

FIG. 21: Structural diagram of Embodiment 3;

FIG. 22: Structural diagram of the magazine holder and magazine of Embodiment 3;

FIG. 23: Structural diagram of Embodiment 3 when the door of equipment housing is opened;

FIG. 24: Internal structural diagram of the first motion platform of Embodiment 3;

FIG. 25: Structural diagram of the first connection part of Embodiment 3;

FIG. 26: Structural diagram of the synchronized connector locking mechanism of Embodiment 3;

FIG. 27: Structural schematic diagram of the needle pulling driver of Embodiment 3;

FIG. 28: Enlarged schematic diagram at position L in FIG. 27;

FIG. 29: First structural diagram of the synchronized connector locking mechanism and synchronized stylet locking mechanism of Embodiment 3;

FIG. 30: Second structural diagram of the synchronized connector locking mechanism and synchronized stylet locking mechanism of Embodiment 3;

FIG. 31: Structural diagram of the seed implantation into the target body in Embodiment 3;

FIG. 32: Internal structural diagram of the wheeled storage mechanism of Embodiment 3;

FIG. 33: Structural diagram of Embodiment 5;

FIG. 34: Structural diagram at the Y-shaped tube in FIG. 33;

FIG. 35: Structural schematic diagram at the outer tube base on the outer tube and the connection holes on the first connection part in FIG. 33.

### DETAILED DESCRIPTION OF THE PREFERRED EMBODIMENT

Further instructions are giver below referring to the drawings and examples, which are not intended to limit the invention.

### Embodiment 1

A radioactive source delivery system comprises a radioactive source delivery mechanism, a stylet pulling mechanism. The radioactive source delivery mechanism includes a wire output channel, a pushing wire, and a wire driving mechanism. The wire driving mechanism connects to the wire output channel, which is used to drive the pushing wire to move back and forth along the wire output channel. The wire output channel is a rigid structure or a flexible structure that can be bent. The stylet pulling mechanism arranged in parallel with the front end of the wire output channel. The stylet pulling mechanism includes a friction stylet pulling component. A part of the friction stylet pulling component presses tightly against the stylet, so the needle stylet is pulled by the frictional force generated by the pressing.

The friction stylet pulling component is one or a combination of a friction wheel component, a friction belt component, or a reciprocating clamping component. The friction wheel component or friction belt component includes one or more sets of friction wheels or friction belts. One side of which can be in close contact with the stylet. The rotation of the friction wheels or the cyclical movement of the friction belts drives the stylet to be pulled out. The reciprocating clamping component comprises a reciprocating motion component and a clamping component. The clamping component is mounted on the reciprocating motion component and can move back and forth along a certain trajectory driven by the reciprocating motion component. The clamping component can clamp the stylet when driven in the direction of the stylet pulling by the reciprocating motion component, pulling out the stylet. Conversely, when driven in the opposite direction by the reciprocating motion component, the clamping component releases the stylet, then resetting.

It further comprises the flexible delivery tube 24018 with a stylet inside. The front end of the stylet extends from the flexible delivery tube, and fills the space inside the trocar needle 18 connected with the front end of the flexible delivery tube, which prevents blood from flowing into the trocar needle, otherwise blood coagulation can lead to the blockage of the trocar needle. The rear end of the stylet extends from the rear end of the flexible delivery tube as a stylet tail portion, and the stylet is equipped with a stop step. The stop step is in contact with rear end of the flexible delivery tube, serving as a positioning function to prevent the other end of the stylet from protruding out of the trocar needle and piercing into the target body 1002.

The stylet pulling component docks with the stylet tail portion inside one of the flexible delivery tubes through a channel-switching mechanism, and to pull out the stylet from the flexible delivery tube, thereby forming a hollow flexible delivery channel. The flexible delivery tube is the first flexible delivery tube. The stylet is a flexible wire with elasticity, which can be bent under the external force and can be restored to a straight state after the external force is removed. The material of the stylet is one or a combination of several types, including Ni—Ti alloy, spring steel, and composite materials. The length of the stylet is greater than 600 mm.

The pushing wire is a flexible pushing wire, and the wire driving mechanism is a flexible wire driving mechanism. The flexible pushing wire is a flexible wire with elasticity, which can be bent under the external force and can be restored to a straight state after the external force is removed. The material of the flexible pushing wire is one or a combination of several types, including Ni—Ti alloy, spring steel, and composite materials. The length of the flexible pushing wire is greater than 600 mm.

The wire output channel docks to one of the flexible delivery tubes through a channel-switching mechanism. The front end of the flexible delivery tube is connected with a trocar needle or has a quick connector designed to connect with a trocar needle. The quick connector is fixedly connected to the trocar needle using one or a combination of thread, latch, and adhesive. The radioactive source delivery mechanism also includes a radioactive source feeding mechanism, which is used to set a radioactive source at the front of the pushing wire. The wire driving mechanism drives the pushing wire forward to push the radioactive source along the wire output channel, through the flexible delivery tube, and into the target body via the trocar needle that has been inserted into the target body.

It includes a first motion platform and a first connecting portion. The first motion platform with one end of the wire output channel and the first connecting portion respectively installed on the opposite sides. The first motion platform is one of the following modes:
A. The first connecting portion moves and one end of the wire output channel remains stationary;
B. The first connecting portion remains stationary and one end of the wire output channel moves;
C. The first connecting portion moves and one end of the wire output channel also moves.

The first motion platform includes a planar motion mechanism and a first front-and-back docking mechanism. The first front-and-back docking mechanism connects to the planar motion mechanism. The planar motion mechanism is used to drive the first front-and-back docking mechanism to move within a plane. The first front-and-back docking mechanism drives one end of the wire output channel or the first connecting portion to move back and forth in a direction perpendicular to that plane.

The planar motion mechanism takes rotational movement in one direction to achieve the motion of the first front-and-back docking mechanism in a plane. Alternatively, the rotary motion in one direction and the linear motion at least in one direction of the planar motion mechanism drives the motion of the first front-and-back docking mechanism in a plane. Alternatively, the planar motion mechanism takes the linear motion in two directions to achieve two degrees of freedom motion of the first front-and-back docking mechanism in space.

It further comprises a second motion platform. The stylet tail portion protrudes from the connection hole of the first connection part. The second platform with a stylet pulling mechanism and a first connection part respectively installed on the opposite sides, which is used to achieve the relative motion in space of the stylet pulling component and the first connection part. It makes the stylet pulling component docks with the stylet tail portion inside any of the flexible delivery tubes attached to the first connection part, performing the pulling of the stylet, pulling it out from the flexible delivery tube, thereby achieving multi-channel stylet pulling. The second motion platform is one of the following modes:
A. The first connection part moves while the stylet pulling component remains stationary;
B. The first connection part remains stationary while the stylet pulling component moves;
C. The first connection part moves while the stylet pulling component also moves.

Alternatively, the second motion platform is the first motion platform. In this scenario, the stylet pulling component and one end of the wire output channel are installed at the same side of the first motion platform. The first motion platform also includes a second front-and-back docking mechanism which connects to the planar motion mechanism. The first front-and-back docking mechanism and the second front-and-back docking mechanism are respectively used for the back and forth docking motion of one end of the wire output channel with the stylet pulling component. First, the stylet pulling component docks with the stylet tail portion inside one of the flexible delivery tubes on the first connection part, and pulls the stylet. After the stylet pulling is completed, a new implantation channel is established. The first motion platform drives the wire output channel connects with the flexible delivery tube, and then the implantation is carried out through the newly established implantation channel.

The stylet pulling component is installed on the first motion platform through a quick connecting structure, which is one or a combination of a threaded connecting structure, a latch connecting structure, or a lock connecting structure.

The first connecting portion connects to the first connection part which has a plurality of connection holes. The one end of each flexible delivery tube is installed at the corresponding connection hole. The first motion platform is used to achieve the docking between one end of the wire output channel with each flexible delivery tube on the first connection part, forming a delivery channel for radioactive source, achieving multi-channel delivery. The first connecting portion is one or a combination of an adhesive connection portion, a welding connection portion, a threaded connection portion, a snap-fit connection portion, or a locking connection portion.

It further comprises a stylet storage mechanism. The stylet storage mechanism is used to store the stylets pulled out by the stylet pulling component and located at the rear end of the stylet pulling component. When the stylet is output from the rear end of the stylet pulling component, the stylet storage mechanism stores the flexible stylet accordingly. Alternatively, the stylet storage mechanism is a part of the stylet pulling mechanism, completing the storage of the stylet while pulling the stylet.

The stylet storage mechanism is a wheeled storage mechanism or a storage tube.

The wheeled storage adopts a wheel-shaped stylet container which has a concave inner surface and has an opening on the side. The stylet extends into the concave inner surface through the side opening. The wheel-shaped stylet container is designed to rotate freely or be actively driven, positioned behind the stylet pulling mechanism. Under the action of the stylet's own elasticity, the stylet is automatically wound within the concave inner surface of the wheel-shaped stylet container. As the stylet is output from the rear end of the stylet pulling mechanism, it drives the wheel-shaped stylet container with a concave inner surface to rotate around the axis of the wheel-shaped stylet container, thereby achieving dynamic storage.

The stylet storage tube can be any one of a straight tube, a spiral tube, or a thin-film tube. The material of the stylet storage tube is one or a combination of metal, plastic, rubber, latex, silicone, or elastomer material. The storage tube contains a lubricant to facilitate the smooth insertion of the stylet, or the inner surface of the storage tube can be evenly coated with grease or the inner surface of the storage tube can take a lubricating coating to achieve the lubricating effect, and the material of the lubricating coating is Teflon. The inlet end of the storage tube has an elastic extension section, which can shorten under the action of press and automatically extend to its original position after the press is released.

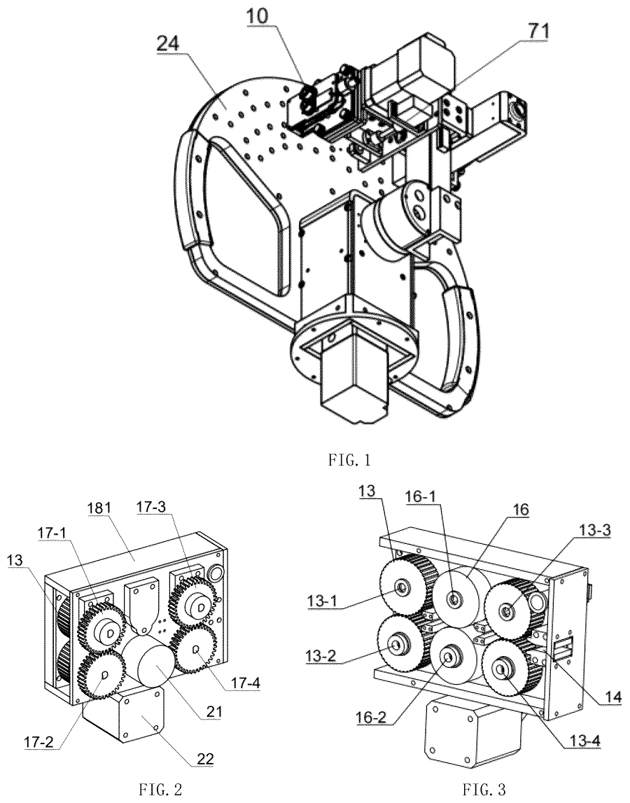

Specifically, FIGS. 1-5 shows the first stylet pulling mechanism 10 including a first motion platform 71 and a first connection part 24. One end of multiple flexible delivery tubes with a flexible stylet inside is installed on the first connection part 24, with the tail end of the flexible stylet exposed from the other side of the first connection part. The first stylet pulling mechanism 10 and the first connection part are installed respectively at opposite sides of the first motion platform 71. The first motion platform 71 enables relative movement between the first stylet pulling mechanism 10 and the first connection part in space, as well as the position/distance adjustment between the first stylet pulling mechanism 10 and different flexible stylet tails. The first motion platform 71 allows the first stylet pulling mechanism 10 to dock with the different flexible stylet tails and pull out the stylet, realizing multi-channel stylet pulling. The first motion platform 71 is equipped with a back-and-forth mechanism, with a storage tube as the stylet storage device. The back-and-forth mechanism can change the relative distance between the first stylet pulling mechanism 10 and the flexible delivery tube. As the back-and-forth mechanism moves forward, the first stylet pulling mechanism 10 docks with the stylet. The first stylet pulling mechanism 10 pulls out the stylet and sends it into the storage tube. During the stylet pulling out, the back-and-forth mechanism moves backward. After the stylet is completely pulled out, the back-and-forth mechanism feeds forward again. Since most of the stylet stays inside the storage tube at this time, the friction between the stylet and the storage tube will cause the front end of the pulled stylet to detach from the tail end outlet of the first stylet pulling mechanism 10, thereby preventing the just-pulled stylet from clogging the first stylet pulling mechanism, facilitating the sequential pulling of multiple stylets.

The back-and-forth mechanism uses a motor to drive the screw to rotate, with the screw pushing the entire first stylet pulling mechanism 10 forward and backward. Alternatively, the back-and-forth mechanism 71 can use belt transmission, gear rack, hydraulic push rod, or pneumatic push rod as substitutes.

The first stylet pulling mechanism 10 employs a friction stylet pulling mechanism, with traction components including multiple friction wheels 13 or multiple friction belts. A movement path 14 is provided between the friction wheels 13 or the friction belts. The friction wheels 13 or the friction belts contact the stylet inside the flexible delivery tube, driving the stylet to move within the movement path 14, thus pulling the entire stylet out of the flexible delivery tube.

As shown in FIGS. 1-4, the position measurement component includes one or more measuring wheels 16, set on one side of the movement path 14. The measuring wheel 16 measures the movement amount when the stylet moves within the movement path 14. The outer surface of the measuring wheel contacts the stylet. When the stylet passes by the side of the measuring wheel 16, it drives the measuring wheel 16 to rotate. The position measurement component also includes a travel switch, which is a conductive travel switch. Utilizing the conductive nature of the stylet, it determines the position of the stylet based on conductive on-off, including elastic contacts or probe pins.

Alternatively, the travel switch can be a mechanical travel switch, photoelectric travel switch, inductive proximity switch, capacitive proximity switch, or magnetic proximity switch.

As shown in FIGS. 1-4, a synchronized transmission mechanism is provided between the multiple friction wheels 13 or the multiple friction belts, ensuring that the friction wheels 13 or friction belts rotate synchronously, achieving stable driving of the stylet. The synchronized transmission mechanism employs one or a combination of belt drive mechanisms, gear transmission mechanisms, and chain transmission mechanisms. A guide channel is set between adjacent sets of friction wheels or friction belts, through which the stylet passes, playing a guiding role to prevent the stylet from bending between adjacent sets of friction wheels or friction belts, which could cause a driving blockage.

One end of the first friction wheel 13-1 is connected to the first gear 17-1. The second friction wheel 13-2 is set below the first friction wheel 13-1. One end of the second friction wheel 13-2 is connected to the second gear 17-2. The first gear 17-1 meshes with the second gear 17-2. The other end of the second friction wheel 13-2 is connected to the first pulley 19-1. One end of the third friction wheel 13-3 is connected to the third gear 17-3. The fourth friction wheel 13-4 is set below the third friction wheel 13-3. One end of the fourth friction wheel 13-4 is connected to the fourth gear 17-4. The third gear 17-3 meshes with the fourth gear 17-4. The other end of the fourth friction wheel 13-4 is connected to the second pulley 19-2. The first pulley 19-1 and the second pulley 19-2 are connected by the first belt 20-1. Below the first measuring wheel 16-1, a second measuring wheel 16-2 is set, with one end of the second measuring wheel connected to the encoder 21. The second housing 181 also has a first motor 22, with the output end of the first motor 22 connected to the second pulley 19-2 by the second belt 20-2.

The friction stylet pulling mechanism also includes a reciprocating mechanism or a triggering mechanism or an active storage mechanism. By using these mechanisms, the stylet is removed from the stylet pulling channel of the stylet pulling mechanism, clearing the stylet pulling channel to avoid the blockage issue during multi-stylet pulling. The reciprocating mechanism changes the distance between the storage device and the stylet pulling mechanism. The entrance of the storage device is equipped with a spring tube component, which can guide the stylet smoothly into the storage device. When the distance between the storage device and the stylet pulling mechanism is shortened, it can compress the spring tube component.

The movement form of the reciprocating mechanism is as follows:
A, driving the stylet pulling mechanism to move back and forth, with the storage device remaining fixed;
B, keeping the stylet pulling mechanism fixed, and driving the storage device to move back and forth. The reciprocating mechanism is one or a combination of screw nut mechanism, gear rack mechanism, belt drive mechanism, pneumatic push rod, or hydraulic push rod.

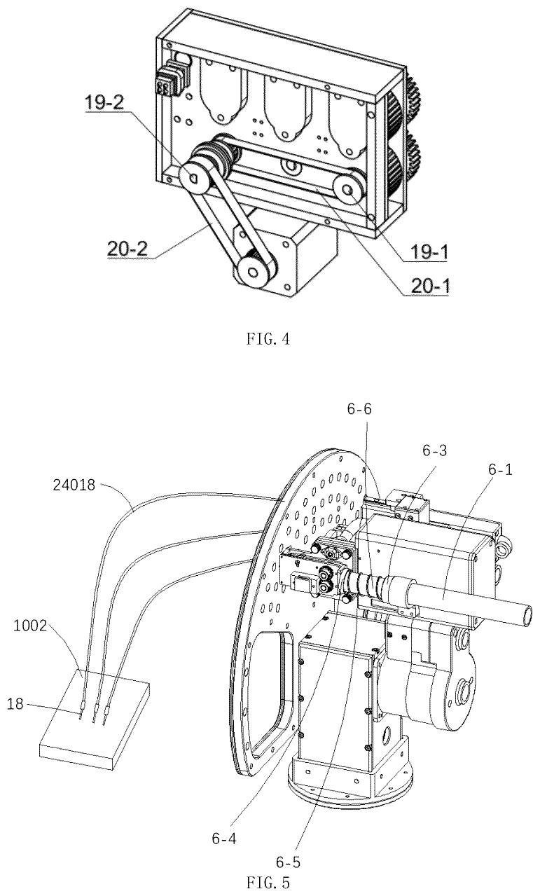

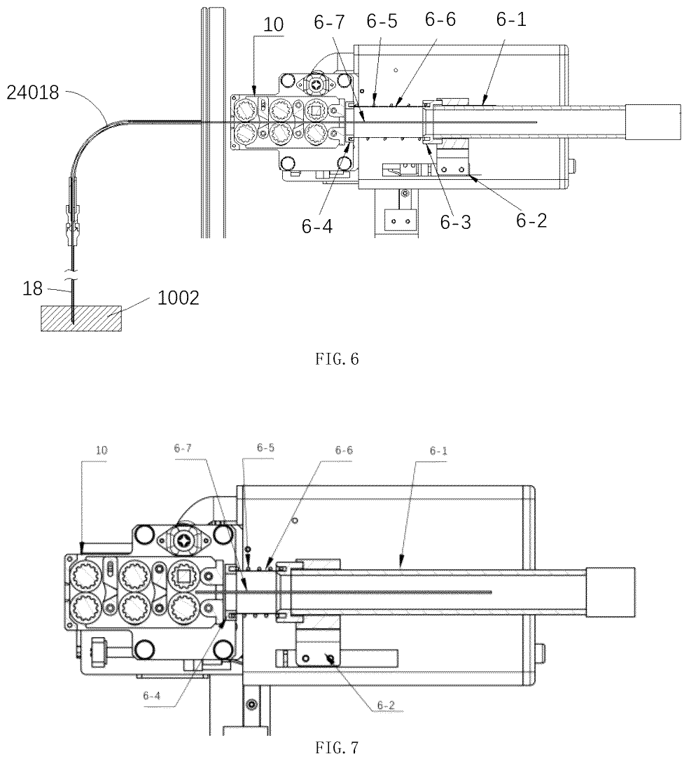

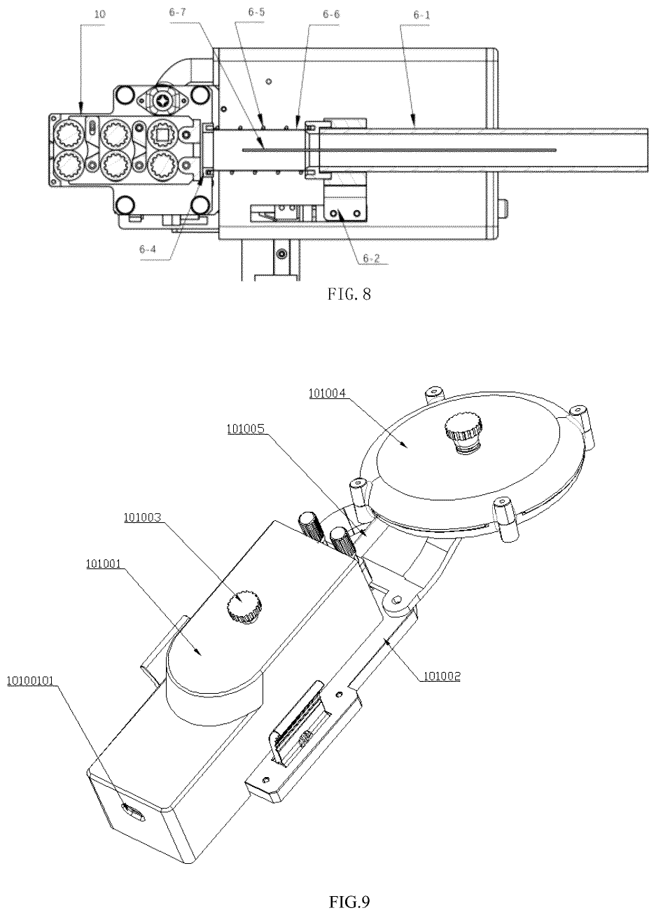

Specifically, as shown in FIGS. 5-8, the reciprocating mechanism changes the distance between the storage device and the first stylet pulling mechanism 10. The storage device 6-1 is limited within the storage device connecting seat 6-2, which is fixed on the fixed part of the reciprocating mechanism. The front end of the storage device 6-1 is equipped with a rear fixing ring 6-3, and a front fixing ring 6-4 is set behind the first stylet pulling mechanism 10. A second spring 6-5 is fixedly connected between the rear fixing ring 6-3 and the front fixing ring 6-4. Inside the second spring 6-5, there is a flexible film 6-6 or a second tube. The second tube can be inserted into the storage tube, or the storage tube can be inserted into the second tube.

The front fixing ring 6-4 is located behind the first stylet pulling mechanism 10. The first stylet pulling mechanism 10 pulls the stylet 6-7 out from the flexible delivery tube and stores the stylet 6-7 through the storage device 6-1. During the process of pulling back the stylet, the entire first stylet pulling mechanism 10 will also move backward, pushing the front fixing ring 6-4 to move backward. When the stylet 6-7 is fully pulled by the first stylet pulling mechanism 10, the first stylet pulling mechanism 10 will move forward again, while the front fixing ring 6-4 returns to its original position by the second spring 6-5. Because most of the collected stylet remains inside the storage tube, the friction between it and the inner wall of the storage tube will cause it to detach from the stylet pulling channel of the first stylet pulling mechanism 10, clearing the stylet pulling channel of the first stylet pulling mechanism 10, and leaving space for the next stylet pulling, avoiding blockages.

### Embodiment 2

A radioactive source delivery system with a stylet pulling mechanism, where the parts of this embodiment that is identical to those in Embodiment 1 will not be described again. The differences are as follows:

It further comprises a stylet insertion mechanism. The stylet insertion mechanism can insert the stylet along the flexible delivery tube into the trocar needle, filling the space inside the trocar needle, which prevents blood from flowing into the trocar needle, otherwise blood coagulation can lead to the blockage of the trocar needle.

The stylet pulling component is also the stylet insertion mechanism, which adopts a friction stylet pulling component. A part of this component presses tightly against the stylet. The friction stylet pulling component is capable of being driven to rotate in both directions, and it uses the frictional force generated by pressing to achieve the extraction and insertion of the stylet.

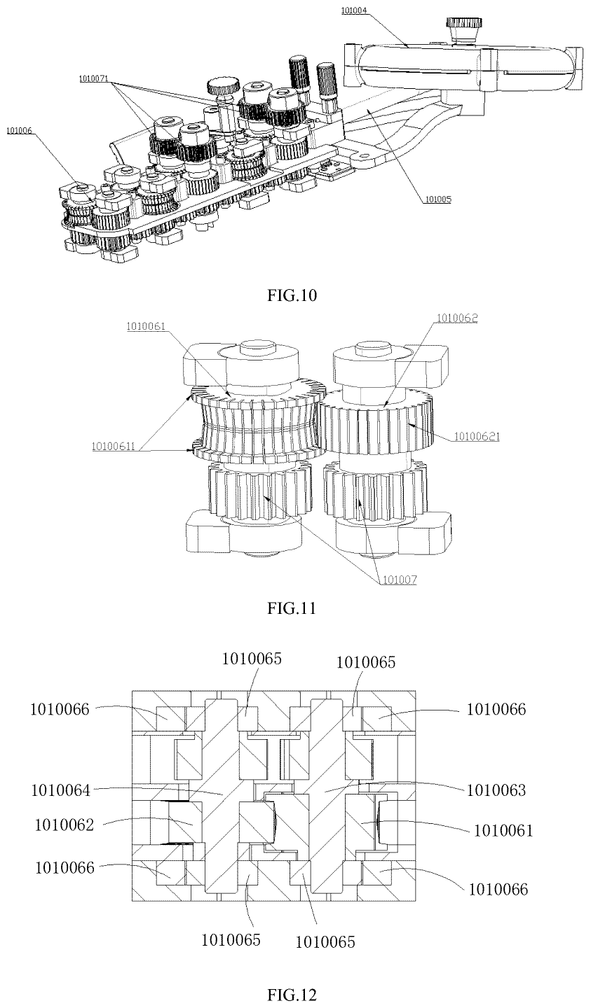

As shown in FIGS. 9 to 15, the stylet pulling mechanism is also the stylet insertion mechanism. The stylet pulling mechanism includes a housing (101001) and a mounting plate (101002). The housing (101001) is mounted on the mounting plate (101002) with hand-tightened screws (101003). The front end of the housing (101001) is provided with an opening (10100101) for the stylet to pass through, and the rear end of the housing (101001) is provided with a storage wheel (101004) for storing the stylet, with a guide tube (101005) for guiding the stylet arranged between the friction stylet pulling component and the storage wheel (101004).

The mounting plate (101002) is equipped with multiple sets of friction wheel sets (101006). Each friction wheel set (101006) includes a first friction wheel (1010061) and a second friction wheel (1010062), both of which have several anti-slip grooves (10100621) circumferentially arranged to effectively increase the friction between the friction wheels and the stylet, preventing the friction wheel set (101006) from slipping while pulling the stylet. Both the upper and lower ends of the first friction wheel (1010061) are provided with a retaining ring (10100611) with a diameter larger than the friction working outer surface, blocking the upper and lower ends of the second friction wheel (1010062) to prevent the stylet from getting stuck on the end surface of the friction wheel set (101006). The outer surface of the first friction wheel (1010061) and/or the second friction wheel (1010062) is provided with an annular groove consisting of upper and lower conical surfaces, each with a taper less than 0.2, allowing the friction wheel set (101006) to drive the stylet while confining its movement within the annular groove, effectively preventing stylet deflection during stylet movement and deviation from the pulling direction. The upper or lower ends of the first friction wheel (1010061) and the second friction wheel (1010062) connect to gears A (101007), and the gears A (101007) below each set of first friction wheel (1010061) and second friction wheel (1010062) are spaced apart and do not mesh. Gears A (101007) between different friction wheel sets (101006) are meshed and transmit power through a set of Gear C (10100701), so the rotation direction of different friction wheel sets (101006) is the same.

The first friction wheel (1010061) and the second friction wheel (1010062) use a pressing mechanism to press the stylet, which can be a passive or active pressing mechanism. Alternatively, the first friction wheel (1010061) and/or the second friction wheel (1010062) are elastic structures that achieve stylet pressing through self-compression.

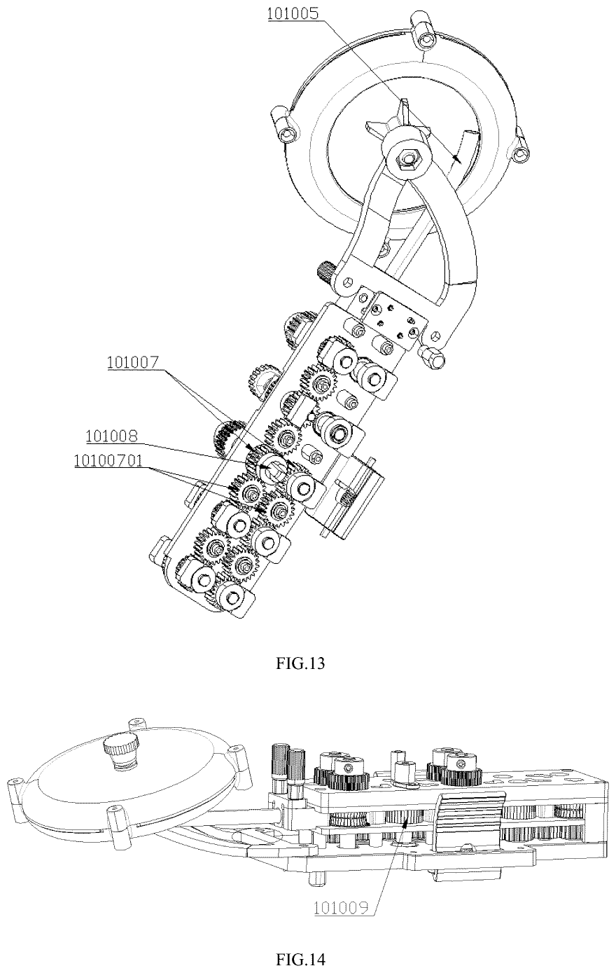

When using a passive pressing mechanism, the passive pressing mechanism presses the stylet by the elastic deformation of the elastic element. As shown in FIG. 12, in this embodiment, the passive pressing mechanism presses the stylet through the elastic deformation of the elastic element. The first friction wheel (1010061) is mounted on the first friction wheel shaft (1010063), and the second friction wheel (1010062) is mounted on the second friction wheel shaft (1010064). The upper and lower ends of the first friction wheel shaft (1010063) are mounted in the long slot of the mounting plates via bearings (1010065), with an elastic element (1010066) arranged between the bearing and the mounting plates in the long slot. The upper and lower ends of the second friction wheel shaft (1010064) are similarly mounted in the long slot of the housing (101001) via bearings (1010065), with an elastic element (1010066) arranged between the bearing and the mounting plates in the long slot. When the stylet is inserted between the first friction wheel (1010061) and the second friction wheel (1010062), both wheels move in opposite directions from the stylet, causing the upper and lower ends of the first friction wheel shaft (1010063) and the second friction wheel shaft (1010064) to squeeze the elastic elements (1010066) and under the elastic force of the elastic elements (1010066), the first friction wheel (1010061) and the second friction wheel (1010062) can press the stylet.

In some friction wheel set (101006), the upper ends of the first friction wheel (1010061) and the second friction wheel (1010062) of each set are equipped with gear B (1010071), and a pair of gear B (1010071) engage with each other. So the rotation direction of gear B (1010071) of each group is opposite, so the first friction wheel (1010061) and the second friction wheel (1010062) rotate in opposite directions. One friction wheel set (101006) is drive connected with an external stylet pulling drive motor through a docking shaft (101008) to achieve power input.

The stylet pulling mechanism also has a measuring wheel (101009), which is not connected with gear A (101007) or gear B (1010071). When the first friction wheel (1010061) and the second friction wheel (1010062) of different groups rotate synchronously, the measuring wheel (101009) does not rotate. Only when the stylet moves relative to the stylet pulling mechanism, the measuring wheel (101009) can be driven to rotate by the friction of the stylet. The measuring wheel (101009) connects to an encoder or with a magnetic element. When a magnetic element is installed, the magnetic element is aligned with an encoder to detect the rotation angle of the measuring wheel (101009), thereby calculating the movement length of the stylet. The measuring wheel (101009) is equipped with a braking mechanism, which quickly stops the measuring wheel (101009) when the stylet does not pass through the measuring wheel (101009) to prevent measurement errors caused by idle rotation. The braking mechanism includes a brake block (101011) and a second elastic element (101010), with the second elastic element (101010) pushing the brake block (101011) against the rotating shaft (1010091) connected to the measuring wheel (101009), or directly against the measuring wheel (101009), controlling the measuring wheel (101009) to stop quickly by friction between the brake block (101011) and the rotating shaft (1010091) or the measuring wheel (101009). The brake block (101011) can also be omitted, with the second elastic element (101010) directly pressing against the rotating shaft (1010091) or the measuring wheel (101009). The second elastic element (101010) can be a spring, a leaf spring, or an elastic block, or a combination of these.

Before using the stylet pulling mechanism, the entire stylet is allowed to pass through the measuring wheel (101009) once. Since the length of the stylet is known, the actual coefficient between the rotation angle of the measuring wheel (101009) and the movement length of the stylet can be calculated through the encoder's measurement results. This actual coefficient can be used to calculate the stylet movement during the subsequent surgical procedure. The actual coefficient can be stored in a chip inside the stylet pulling mechanism (this chip communicates with the host and receives power and data via conductive contacts). The next time it is used, the parameter can be read from the chip and used as the initial coefficient, effectively preventing inaccuracies in stylet displacement measurement due to wear of the measuring wheel (101009) over long-term use.

A group of friction wheels is also installed behind the measuring wheel (101009). When the stylet passes through the measuring wheel (101009) once and exits, the brake block (101011) quickly stops the measuring wheel (101009) to rotate. After the encoder detects that the measuring wheel (101009) has stopped rotating, the stylet pulling drive motor stops. Then, only the group of friction wheels behind the measuring wheel (101009) can drive the stylet. When it is necessary to eject the stylet, the stylet pulling drive motor rotates in reverse, causing all friction wheels to rotate in reverse and driving the stylet backward through the group of friction wheels behind the measuring wheel (101009) until the stylet is completely ejected from the stylet pulling mechanism. Since the length of the stylet is known, the outer diameter of the measuring wheel 101009 (actual coefficient between the rotation angle of the measuring wheel 101009 and the movement length of the stylet) can be calculated by measuring the rotation angle of the measuring wheel 101009.

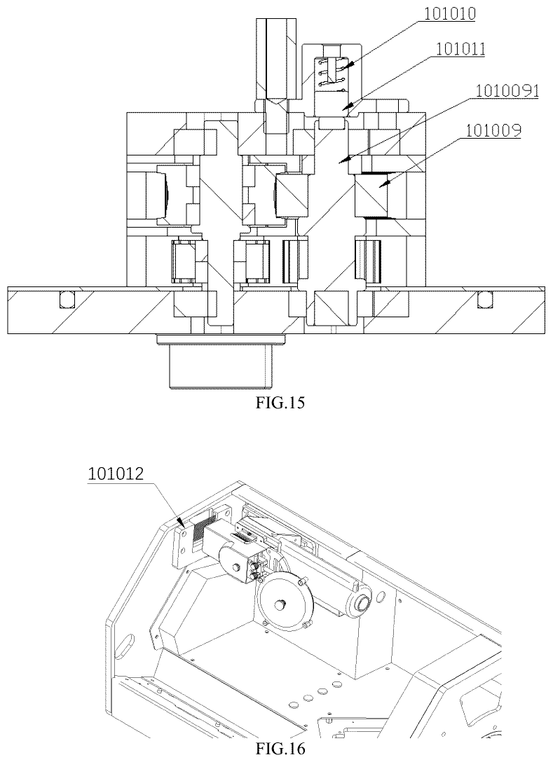

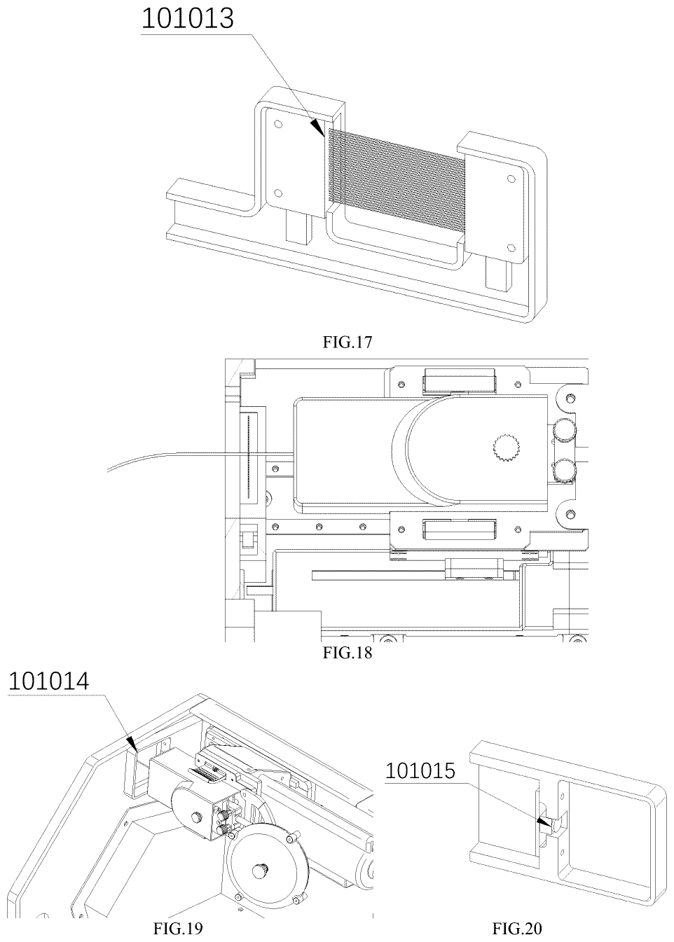

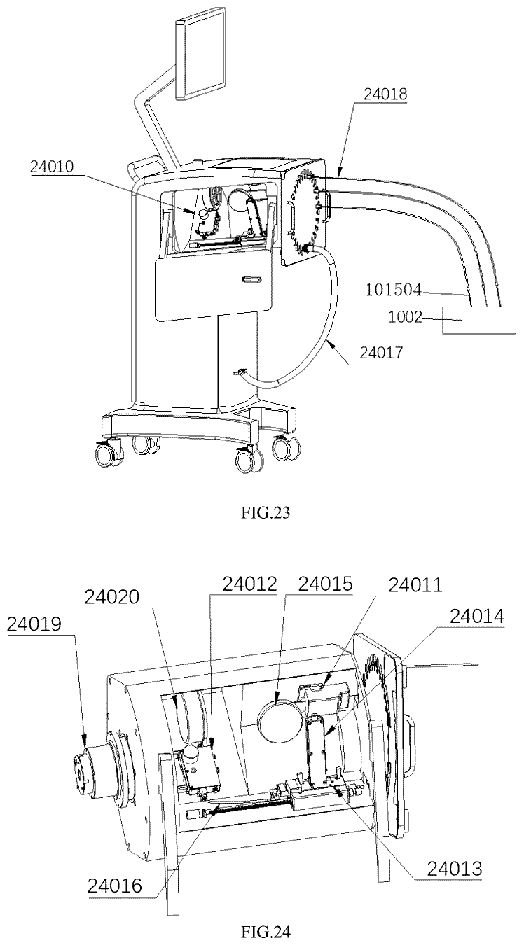

As shown in FIGS. 16 to 20, the stylet pulling mechanism is installed on a back-and-forth mechanism inside a drum. The front end of the drum is equipped with a first connection part, with multiple flexible delivery tubes mounted on the first connection part. The flexible delivery tubes contain the stylet. The rotation of the drum and linear motion of the back-and-forth mechanism drive the stylet pulling mechanism to dock with the stylet inside different flexible delivery tubes, pulling out the stylet from the flexible delivery tubes or reinserting the pulled stylet back into the flexible delivery tubes. Alternatively, the drum and the back-and-forth mechanism drive the stylet pulling mechanism to dock with the stylet storage tube on the first connection part (see stylet storage tube 24017 in FIG. 23), sending the pulled stylet into the stylet storage tube. A laser detection module (101012) will be installed in front of the stylet pulling mechanism, fixed to the drum. The laser detection module (101012) contains a laser sensor (101013). When the stylet pulling mechanism needs to reinsert the stylet, the entire stylet pulling mechanism moves forward and then ejects the internal stylet. After completion, the stylet pulling mechanism moves backward, using the laser detection module (101012) to determine whether a stylet exists between the stylet pulling mechanism and the flexible delivery tube or stylet storage tube on the first connection part. If no stylet is detected, it indicates that the stylet pulling mechanism has completely ejected the stylet; if a stylet is detected, it indicates that the stylet pulling mechanism has not fully ejected the stylet and triggers an error alarm.

Similarly, the laser detection module (101012) can also be replaced by a camera module (101014). The camera module (101014) contains a camera sensor (101015) used to determine whether there is a stylet between the stylet pulling mechanism and the flexible delivery tube or stylet storage tube on the first connection part.

### Embodiment 3

A radioactive source delivery system with a stylet pulling mechanism is provided, the parts of this embodiment that have the same structure as those of Examples 1 to 2 are not described in detail, and differences are as follows.

The radioactive source feeding mechanism uses a magazine for feeding. The radioactive source feeding mechanism (magazine) is directly integrated into the wire output channel. Seeds, or seed strands, or seed strand casings are loaded into the storage slot or hole within the magazine. The feeding mechanism installed on the magazine is used to place seeds, or seed strands, or seed strand casings at the front end of the pushing wire for feeding.

The flexible delivery tube includes an inner tube and an outer tube. The outer tube is sleeved outside the inner tube. The inner tube is configured to be connected with a puncture needle. The inner tube and the outer tube both include a flexible section, with the length of the flexible section being greater than 600 mm. The flexible section is either a plastic tube or a medical braided tube.

The plastic tube is made of a PTFE material.

A first motion platform includes a back-and-forth movement module and a rotational movement module, and the first motion platform realizes the movement of one end of the wire output channel in the space through a one-direction rotational movement and a one-direction linear movement.

When the first motion platform is disposed inside a main body housing and includes the back-and-forth movement module and the rotational movement module, a front side of the main body housing is a connection part panel (first connection portion). The first connection part panel is mounted with a first connection part, the connection part is provided with a plurality of connection holes on a rotational track of the first motion platform. Each connection hole may be connected to a delivery tube, and the other end of the delivery tube is connected with a puncture needle. The motion platform is in a shape of a drum, and a wire driving mechanism is disposed in the drum.

The rotational movement module drives the drum to rotate around the axis of the drum, and the back-and-forth movement module is disposed in the drum for driving one end of the wire output channel to move forward and backward.

A door is disposed on a left and/or the right side of the main body housing, and a combination of a wire driving mechanism, a stylet pulling mechanism, and a magazine base, or a combination of a wire driving mechanism, a stylet pulling mechanism, and a docking tube structure may be mounted on the first motion platform by opening the door. The stylet pulling mechanism, the magazine base, and the docking tube are mounted perpendicular to the first connection part panel, and a magazine is mounted on the magazine base or mounted on the wire driving mechanism.

When the wire driving mechanism and the magazine base are installed on the first motion platform, the wire driving mechanism and the magazine base are connected by a flexible connection tube, and at this time, the flexible connection tube is a part of the wire output channel, and an outlet of the magazine base is a docking head, which is a terminal end of the wire output channel.

When the wire driving mechanism and the docking tube structure are mounted on the first motion platform, the wire driving mechanism and the docking tube structure are connected by the flexible connection tube, and at this time, the flexible connection tube is a part of the wire output channel, an outlet of the docking tube structure is a terminal end of the wire output channel, and the docking tube structure adopts a floating docking tube structure which can float within a certain range.

The flexible delivery tube is quickly mounted to the connection hole of the first connection part by means of a quick connector, and the first connection part is provided with a synchronized connector locking mechanism, which may lock and position the quick connector of each flexible delivery tube simultaneously.

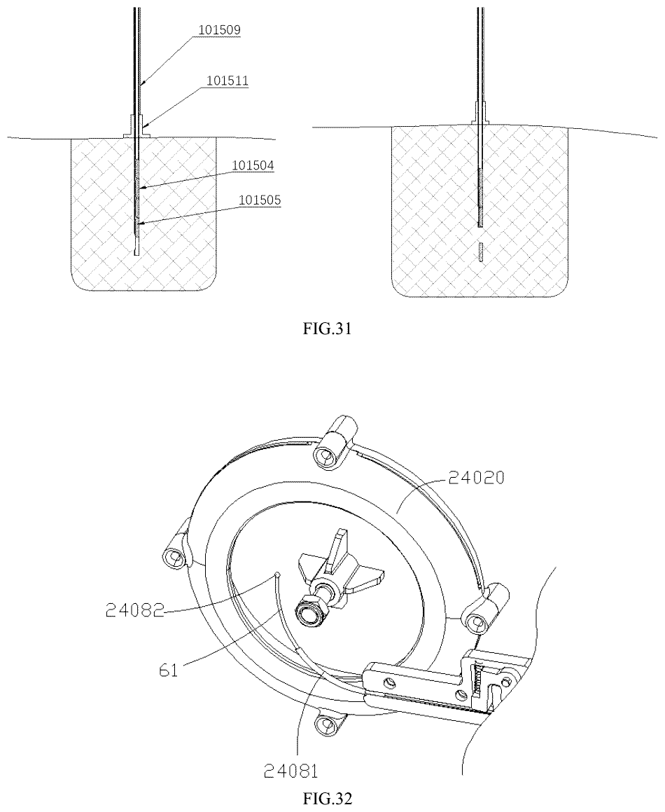

As shown in FIG. 31, the synchronized connector locking mechanism includes a connector clamping plate 24038 disposed within the first connection part, and connector clamping portions are arranged in a circular array on the connector clamping plate 24038. In an unlocked state, all connector clamping portions are staggered with the connection holes, and the quick connector 24029 of the flexible delivery tube can be inserted into the connection holes. When locking, the connector clamping plate 24038 is driven by a clamping driving mechanism to rotate around a center axis, and the connector clamping portions extend into the connection holes. A side of the quick connector 24029 of the flexible delivery tube is provided with a slot, and the quick connector 24029 of the flexible delivery tube is fixed on the first connection part when the connector clamping plate 24038 is rotary to clamp into the slot.

The clamping driving mechanism is electrically or manually actuated and drives the connector clamping plate to rotate around the central axis through cams, gears, or belt drives.

A stylet is disposed inside the flexible delivery tube, extends all the way along the flexible delivery tube, and fills up a space inside the puncture needle connected at a front end of the flexible delivery tube, so as to avoid the blood from pouring into the puncture needle to coagulate and form a clogging. The first connection part is also provided with a synchronized stylet locking mechanism, which may lock and position the stylet inside each flexible delivery tube simultaneously.

As shown in FIG. 30, the synchronized stylet locking mechanism includes a stylet clamping plate 24040 disposed inside the first connection part, stylet clamping portions are arranged in a circular array on the stylet clamping plate 24040. In the unlocked state, all stylet clamping portions are staggered from the connection holes, and the quick connector 24029 of the flexible delivery tube can be inserted into the connection holes. When locking, the stylet clamping plate 24040 rotates around a central axis driven by a stylet driving mechanism, and each of the stylet clamping portions inserts into the slot of each of the connection holes. The slot connects to an elastic body 24041 inside the quick connector 24029. The slot is provided on the side of the quick connector 24029 of the flexible delivery tube, and the elastic body 24041 adopts one or a combination of an elastomer, a rubber tube, or a TPU tube. The elastic body 24041 is adjacent to the stylet, and the stylet clamping plate 24040 presses against the elastic body 24041 during rotation to press down the stylet, thereby realizing the locking.

Alternatively, the synchronized connector locking mechanism is the synchronized stylet locking mechanism, the connector clamping plate is the stylet clamping plate, and the clamping driving mechanism is the stylet driving mechanism, and when the clamping driving mechanism drives the connector clamping plate to rotate around a center axis by a first angle, the connector clamping plate inserts into the slot of the quick connector of the flexible delivery tube while not holding against the elastic body. The connector clamping plate fixes the quick connector of the flexible delivery tube on the first connection part, which just realizes locking of the quick connector of each flexible delivery tube but not realizes locking of the style inside each flexible delivery tube.

When the clamping driving mechanism drives the connector clamping plate to rotate around the center axis by a second angle, the connector clamping plate presses against the elastic body during rotation, and then presses the stylet, which realizes locking of the quick connector and the style inside the each flexible delivery tube.

The stylet needs to be pulled out before an implantation, the stylet is a flexible wire with elasticity, which may be bent under an external force and restored to a straight state after the external force is withdrawn. A length of the stylet is greater than 600 mm. A rear end of the stylet extends backwardly by a portion from a rear end of the flexible delivery tube, and the rear end of the stylet is provided with a first stopping step, and when the first stopping step is pressed against the rear end of the flexible delivery tube, a distance between a front end of the stylet and the front end of the puncture needle connected to the front end of the flexible delivery tube is no more than 5 mm.

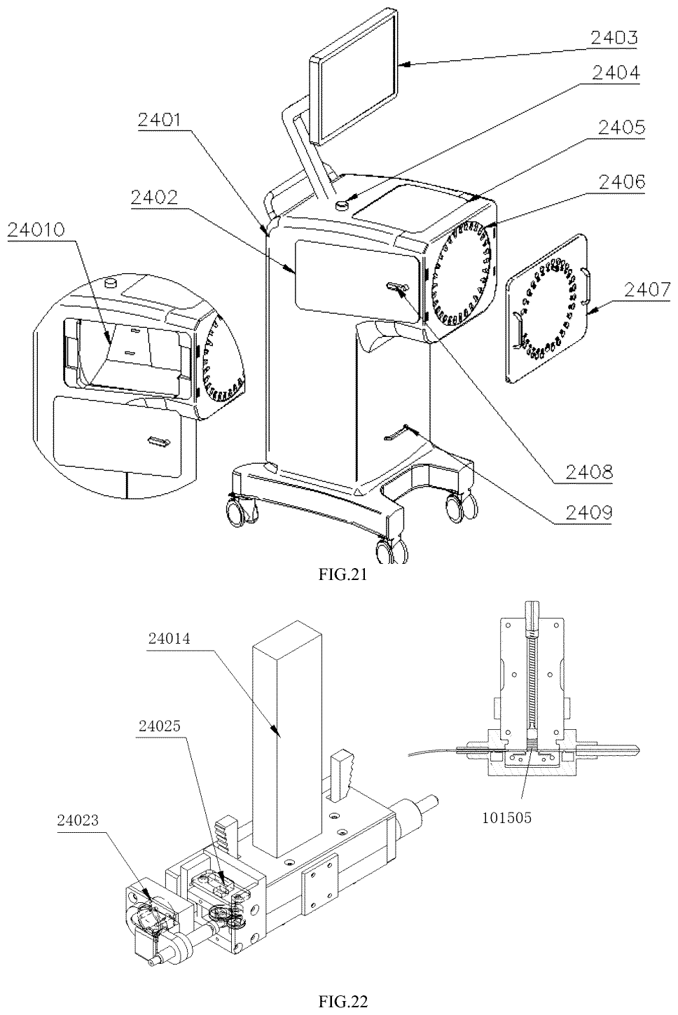

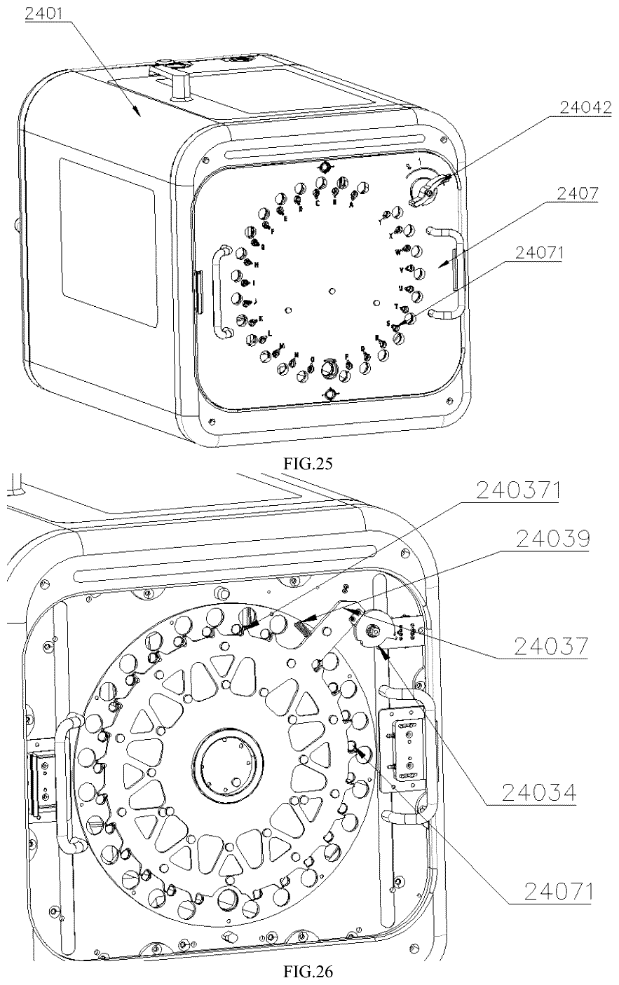

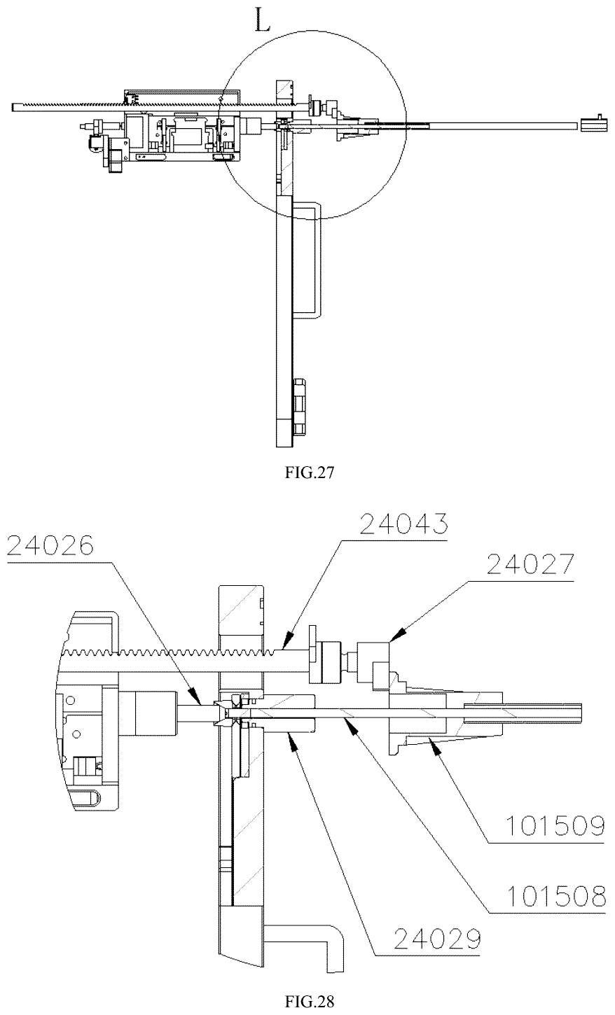

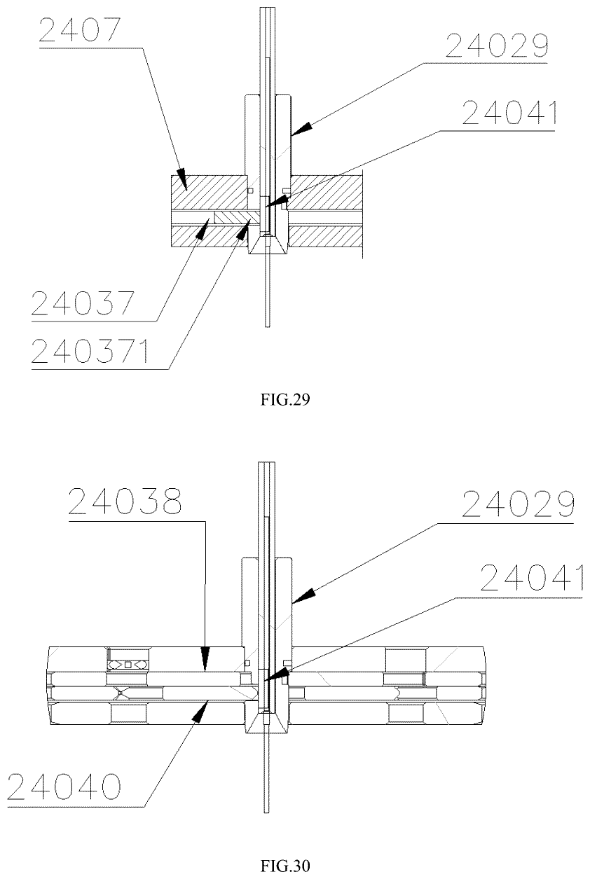

As shown in FIGS. 21-31, the first motion platform is the drum, the drum is disposed inside the main body housing, the drum has a one-direction rotational movement around the axis of the drum. There are an implantation docking mechanism and a stylet pulling docking mechanism inside the drum. And the wire driving mechanism, the stylet pulling mechanism, and the magazine base are provided on the drum. The radioactive source feeding mechanism adopts a magazine, the first connection part adopts a docking plate, and a needle pulling driver drives the inner tube or outer tube to make a relative sliding movement between the inner tube or outer tube by direct pushing and pulling.

A main body of a radioactive source implantation mechanism includes a main body housing 2401, a door 2402, a display screen 2403, an emergency stop button 2404, an observation window 2405, a docking plate panel 2406, a docking plate 2407, a door handle 2408, a support column 2409, and a drum 24010.

Before the surgery, the door 2402 is in a closed state, the door 2402 is opened by rotate the door handle 2408, the door 2402 is located on the left and/or right side of the main body housing 2401, accessories are installed inside the drum 24010. Specifically, the stylet pulling mechanism 24011, the wire driving mechanism 24012, and the magazine base 24013 are installed in corresponding positions in the drum 24010. The stylet pulling mechanism 24011 and the magazine base 24013 are vertically mounted on the docking plate panel 2406 (referring to FIG. 17). A stylet storage wheel 24015 is disposed at the rear side of the stylet pulling mechanism 24011, and a wire storage wheel 24020 is provided at the rear side of the wire driving mechanism 24012, and the flexible pushing wire 61 is stored inside the wire storage wheel 24020, and a main material of the flexible pushing wire 61 is a Ni—Ti alloy wire with a certain degree of toughness. One end of the connection tube 24016 is connected to the wire driving mechanism 24012, the other end of the connection tube 24016 is connected to a force transducer 24023 on the magazine base 24013, and there is a bending section of the connection tube 24016. A magazine 24014 is fitted into the magazine base 24013. The docking plate 2407 is installed on the docking plate panel 2406. A stylet disposal tube 24017 is fixed on a stylet disposal hole of the docking plate 2407. The door 2402 is closed when the installation is complete.

At the beginning of the surgery, a corresponding count of flexible delivery tubes 24018 are prepared in accordance with the count of puncture needles required for the surgery, and quick connectors 24029 of a plurality of flexible delivery tubes 24018 are sequentially installed into connection holes 24071 on the docking plate 2407, a locking handle 24042 is sequentially rotated to a first gear position, a cam 24034 internally connected with the locking handle 24042 rotates to rotate a connector locking plate 24037, the connector locking plate 24037 is provided with a plurality of convex plates 240371 (connector locking portion) corresponding to the connection holes 24071, and the convex plates 240371 rotate to be embedded in a slot on a side of quick connectors 24029, but not hold against the elastic body 24041, thereby fixing the quick connectors 24029 of the flexible delivery tube 24018 to the docking plate 2407. Then the locking handle 24042 is rotated to a second gear position, the cam 24034 internally connected with the locking handle 24042 rotates to cause the connector locking plate 24037 to continue to rotate, and the convex plate 240371 rotates to press against the elastic body 24041 inside the quick connectors 24429, and the elastic body 24041 simultaneously presses against a stylet inside the flexible delivery tube 24018. Then, the mechanism simultaneously locks all the quick connectors 24429 of the flexible delivery tube 24018 and the stylets inside the flexible delivery tube on the docking plate 2407.

On the other side, an operator holds a puncture needle handle and pierces the second puncture needle 101504 into a target surgical position of a target object 1002 during the surgery. After the puncture is completed, a needle stylet inside the second puncture needle (the needle stylet is short in length and is used only for puncture) is pulled out, and then the front end of the long flexible stylet protruding from the front end of the flexible delivery tube is inserted into the second puncture needle to fill the space inside the second puncture needle, and then the rear end of the second puncture needle is connected to a second quick connector at the front end of the flexible delivery tube, forming a complete implantation channel.

Then, turning the locking handle 24042 along an opposite direction to the first gear position, a spring 24039 connected with the connector locking plate 24037 springs back to cause the convex plate 240371 to loosen the elastic body 24041 and the stylet, but still lock the quick connectors 24029.

When a user starts the seed implantation system, the machine starts to operate, a motor 24019 controls an internal drum 24010 to rotate, thereby making the stylet pulling mechanism 24011 move to align with a flexible delivery tube 24018. Then the stylet pulling mechanism 24011 moves forward to pull out the stylet inside the flexible delivery tube 24018 (by docking with the tail portion of the stylet), and the stylet may be delivered to the stylet storage wheel 24015 (a concave structure) on the rear side of the stylet pulling mechanism 24011, the stylet automatically coil inside the stylet storage wheel 24015 under its own elastic action, and drive the stylet storage wheel 24015 to rotate synchronously. After the stylet is completely pulled out of the flexible delivery tube 24018, the stylet pulling mechanism 24011 returns backwardly. The stylet pulling mechanism 24011 is controlled to move back and forth through a linear movement mechanism, and the linear movement mechanism is one or a combination of a screw nut mechanism, an electric pushing rod, and a rack and pinion mechanism. The motor 24019 controls the drum 24010 to rotate so that the stylet pulling mechanism 24011 is aligned with the disposing tube 24017 (aligned with the stylet disposing hole of the docking plate 2407), and the stylet pulling mechanism 24011 then works to completely spit out the stylet and inserts the stylet into the disposing tube 24017 (stylet storage tube).

Subsequently, the motor 24019 drives the drum 24010 to rotate so that the front end of the wire output channel is aligned to the flexible delivery tube 24018 in which the stylet has just been pulled, and then the front end of the wire output channel is docked to the quick connector 24029 of this flexible delivery tube 24018. The front end of the wire output channel is controlled to move back and forth through the implantation docking mechanism, and then the wire driving mechanism 24012 drives the flexible pushing wire inside the wire storage wheel 24020, and the flexible pushing wire pushes out a seed inside the magazine 24014 to the docking tube 24026 at the front of the magazine 24014, and repeats until the wire output channel is piled up with a specified number of seeds, and then all of the seeds in front of the flexible pushing wire are pushed into the flexible delivery tube 24018 until they are delivered into a second puncture needle 101504.

In this scheme, the magazine base 24013 is disposed at the front portion of the wire output channel, a passive measurement wheel is provided inside the magazine base 24013 to press the flexible pushing wire, the friction generated during the movement of the flexible pushing wire drives the passive measurement wheel to rotate, and the rotation angle of the passive measurement wheel is measured by a encoder 24025. A displacement of the flexible pushing wire may be converted by the encoder 24025. Since an encoder module and a measuring wheel are also disposed inside the wire driving mechanism 24012, then readings of the two encoders may be used to determine whether the passive measurement wheel is slipping, so as to ensure the reliability of a measurement result based on two measurement results, and through reciprocal movements, seeds being pushed reach a specified count. Then the wire driving mechanism 24012 drives the flexible pushing wire to push out the seeds through the flexible delivery tube 24018 to the front end of the puncture needle. In this process, if a resistance subjected by the flexible pushing wire becomes larger during movement, its reaction force may be transmitted to the force sensor 24023 connected with the connection tube 24016 and fed back.

In another embodiment, reflective stripes or color stripes are provided on the flexible pushing wire, and a photoelectric sensor is disposed in the magazine base 24013 or the wire driving mechanism 24012, so that when the relative displacement of the flexible pushing wire and the photoelectric sensor occurs, the reflective stripes or color stripes may synchronously generate a pulse signal, and an actual displacement of the flexible pushing wire may be measured by sampling the pulse signal, and this solution can avoid slippage between the passive measurement wheel and the flexible pushing wire that may affect the measurement accuracy and reliability of the encoder.

Subsequently, a needle pulling rod 24043 is pushed forward until it comes into contact with an outer tube base 101509 of outer tube, at which time a contact signal is sensed by a contact sensor 24027, and the contact sensor 24027 is one or a combination of a force sensor, a mechanical switch, an inductive switch, and a photoelectric switch. Then, as shown in FIG. 31, when a plurality of seeds 101505 reach a position of the second puncture needle 101504 connected with the flexible delivery tube, the wire driving mechanism 24012 may push out the first seed into the human body, and then the needle pulling rod 24043 may continue to push the outer tube base 101509 to move forward, and the front end of the outer tube may be pressed against a surface of the body or a puncture template (which may also be an instrument such as a prostate puncture template, etc.), and when the outer tube base 101509 is pushed forward, an inner tube 101508 moves relative to the outer tube and pulls the second puncture needle 101504 connected at the front end of the inner tube partly out of the human body, and then the wire driving mechanism 24012 may continue to push out a next seed 101505 until the plurality of seeds are implanted respectively through repeated operations.

The wire storage wheel 24020 is a wheel-shape container with a concave inner surface (referring to FIG. 32), the wheel-shape container may rotate freely around its axis, and the edge of the wheel-shape container is a concave structure, a storage region is formed at the concave inner surface of the wheel-shape container. At least one side of the wheel-shape container is provided with an opening, and the flexible pushing wire 61 is guided by a storage guiding tube 24081 to extend into the wheel-shape container through the opening on the side of the wheel-shape container.

When the wire driving mechanism 24012 drives the flexible pushing wire to move back and forth, under an elastic action of the flexible pushing wire, the flexible pushing wire is automatically coiled inside a concave inner surface of the wire storage wheel 24020, and the wire storage wheel 24020 rotates along with the back-and-forth movement of the flexible pushing wire, thereby realizing automatic storage.

In other embodiments, a second stopping step 24082 may be added to the rear end of the flexible pushing wire, and a position of the second stopping step is set to satisfy following principles: when the front end of the flexible pushing wire extends from the flexible delivery tube into the puncture needle and probes out from the front end of the puncture needle by more than a certain length (the length is less than 10 mm), the second stopping step is stuck on the rear step of the storage guiding tube, so as to realize the mechanical position-limiting, which prevents the flexible pushing rod from penetrating further into body tissue, thus ensuring overall safety. Preferably, the second stopping step is an end surface of a stopping tube socketed to the rear end of the flexible pushing wire, or the second stopping step is a welded ball at the rear end of the flexible pushing wire. When the wire driving mechanism 24012 drives the flexible pushing wire to move backward again, the flexible pushing wire is automatically coiled inside the inner concave surface of the wire storage wheel 24020 under the elastic action of the flexible pushing wire, and the wire storage wheel 24020 rotates along with the back and forth movement of the flexible pushing wire, thereby realizing automatic storage.

In other embodiments, a wire connection portion may also be provided on the wire storage wheel 24020, the wire connection portion may be configured to connect and fix the rear end or a middle portion of the flexible pushing wire with the wire storage wheel 24020. A position of the wire connection portion may be set to satisfy following principles: when the front end of the flexible pushing wire extends from the flexible delivery tube into the puncture needle and probes out from the front end of the puncture needle by more than a certain length, the flexible pushing wire is pulled by the wire connection portion so that it is unable to continue to move forward, thereby realizing a mechanical position limiting, which avoids the flexible pushing wire from piercing into the body tissues to cause injuries, and ensuring the overall safety. The wire connection portion adopts a form of threaded connection, bonding, welding, snap connection, and other forms to realize connecting and fixing.

### Embodiment 4

The parts of this embodiment that are identical to those in Embodiments 1 to 3 will not be reiterated. The differences are as follows:

The radioactive source feeding mechanism uses a seed strand driving mechanism for feeding seed strands. The radioactive source feeding mechanism comprises a seed strand driving mechanism, a seed strand output channel, and a cutting mechanism. It outputs seed strands or seed strand casings by the seed strand driving mechanism and cuts them to the desired length by the cutting mechanism, to achieve the feeding of seed strands or seed strand casings. When the seed strand driving mechanism outputs a seed strand casing, the radioactive source feeding mechanism also includes a seed embedding mechanism. This seed embedding mechanism is capable of inserting seeds and/or spacers into the seed strand casing from one end or the side, thereby forming a complete seed strand. The seed strand driving mechanism is connected to the seed strand output channel. The seed strand output channel is a rigid structure or a flexible structure that can be bent. The seed strand output channel is connected with a Y-shaped channel or a second channel switching mechanism.

A seed strand driving mechanism is used to push out the seed strand, which is then cut by the cutting mechanism. The flexible pushing wire is used to push the cut seed strand of the target length into the target position. The needle pulling mechanism drives the flexible delivery tube to automatically pull the needle, synchronizing the automatic needle pulling with the implanting of the seed strand. Alternatively, the aforementioned seed strand can also be replaced with seeds, and the radioactive source feeding mechanism can adopt a seed magazine.

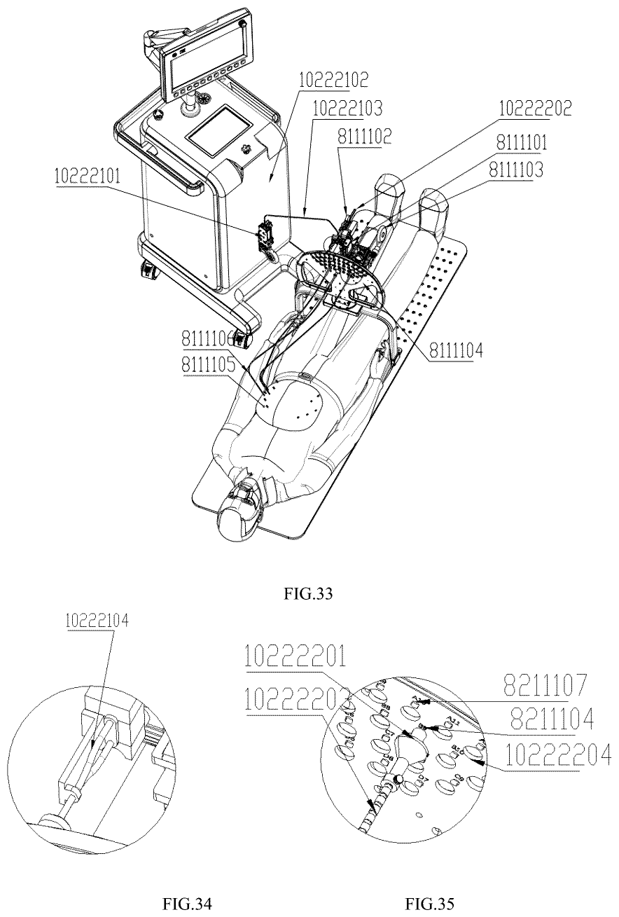

As shown in FIGS. 33 to 35, the flexible delivery tube includes inner tube and outer tube. The outer tube arranged outside the inner tube. The inner tube (not shown in the figures) can slide relative to the outer tube 8111106. One end of the inner tube is fixedly connected to the puncture needle (not shown in the figures), while the other end is fixedly connected to the quick connector 8211104. The quick connector 8211104 is connected to the connection hole 8211107 on the first connection part 8111104. The front end of the outer tube 8111106 is braced against the 3D printed puncture template 8111105. The rear end of the outer tube includes an outer tube base 10222201, which can be manually adjusted to alter its position relative to the main body of the outer tube 8111106. The outer tube base 10222201 can lock the outer tube at different positions. Due to varying initial insertion depths of each puncture needle, the position where the outer tube base 10222201 locks on the outer tube also varies. It is necessary to ensure that the distance between the outer tube base 10222201 and the first connection part 8111104 is relatively small to reserve sufficient needle pulling distance. This distance allows the needle pushing rod 10222202 to push the outer tube base 10222201. One end of the wire output channel 10222103 is connected to the flexible pushing wire driving mechanism 10222101, and the other end is connected to one branch of the Y-shaped tube 10222104. The other branch of the Y-shaped tube is connected to the outlet of the seed strand feeding mechanism 8111102. The two branches of the Y-shaped tube converge into a single outlet, which can align with any connection hole on the first connection part 8111104 under the action of the rotating arm mechanism 8111101. Below each connection hole 8211107 is a corresponding needle pushing hole 10222204. The needle pushing rod 10222202 can pass through the needle pushing hole 10222204 to push the outer tube base 10222201, causing relative movement between the outer and inner tubes. As the front end of the outer tube of the flexible delivery tube presses against to the 3D printed puncture template 8111105, the curvature of the inner tube and outer tube of the flexible delivery tube changes, and the needle retracts relative to the 3D printed puncture template 8111105, pulling the puncture needle partly out of the target body.

The rotating arm mechanism 8111101 will automatically align the entrance of the second stylet pulling mechanism 8111103 with the corresponding connection hole 10222204 for this flexible delivery tube on the first connection part 8111104. The first stylet (not shown in the figures) is then collected into the stylet storage mechanism by the second seed pulling mechanism 8111103. The seed strand feeding mechanism 8111102 cuts a seed strand of the required length and pushes it to the front tube of the Y-shaped tube 10222104. The flexible pushing wire driving device 10222101 then pushes the flexible pushing wire (not shown in the figures) through the pushing wire output channel 10222103 and Y-shaped tube 10222104, pushing the seed strand to the target position. At this point, the needle pushing rod 10222202 pushes the outer tube base 10222201, pulling the puncture needle partly out of the target body, so as to adjust the depth of the needle and control the implantation position of the radioactive source.

The described embodiments are only preferred embodiments of the present invention, and it should be noted that various alterations and improvements may be made therein by those of ordinary skill in the art without departing from the principle of the present invention and should also fall within the scope of protection of the present invention.

Although preferred embodiments have been described herein in detail, it should be noted and will be appreciated by those skilled in the art that numerous variations may be made within the scope of this invention without departing from the principle of this invention and without sacrificing its chief advantages. The terms and expressions have been used herein as terms of description and not terms of limitation. There is no intention to use the terms or expressions to exclude any equivalents of features shown and described or portions thereof and this invention should be defined in accordance with the claims which follow.

We claim:

1. A radioactive source delivery system with a stylet pulling mechanism, comprising:
a radioactive source delivery mechanism including a wire output channel, a pushing wire, and a wire driving mechanism. The wire driving mechanism connects to the wire output channel, which is used to drive the pushing wire to move back and forth along the wire output channel. The radioactive source delivery mechanism also includes a radioactive source feeding mechanism, which is used to set a radioactive source at the front of the pushing wire; and
one or more flexible delivery tubes. Each flexible delivery tube has a flexible stylet inside, the front end of the flexible stylet extends from the front end of the flexible delivery tube and fills the space of the trocar needle which is connected to the front end of the flexible delivery tube. This prevents blood from flowing into the trocar needle, otherwise blood coagulation can lead to the blockage of the trocar needle; and
a stylet pulling mechanism arranged in parallel with the front end of the wire output channel. The stylet pulling mechanism includes a friction stylet pulling component.

2. A radioactive source delivery system with a stylet pulling mechanism according to claim 1, wherein the friction stylet pulling component is one or a combination of a friction wheel component, a friction belt component, or a reciprocating clamping component. A part of the friction stylet pulling component presses tightly against the flexible stylet, so the needle stylet is pulled by the frictional force generated by the pressing. The friction wheel component or friction belt component includes one or more sets of friction wheels or friction belts. One side of which can be in close contact with the flexible stylet. The rotation of the friction wheels or the cyclical movement of the friction belts drives the flexible stylet to be pulled out. The reciprocating clamping component comprises a reciprocating motion component and a clamping component. The clamping component is mounted on the reciprocating motion component and can move back and forth along a certain trajectory driven by the reciprocating motion component. The clamping component can clamp the flexible stylet when driven in the direction of the flexible stylet pulling by the reciprocating motion component, pulling out the flexible stylet. Conversely, when driven in the opposite direction by the reciprocating motion component, the clamping component releases the flexible stylet, then resetting. Repeat the above procedure until the flexible stylet is completely pulled out.

3. A radioactive source delivery system with a stylet pulling mechanism according to claim 1 further comprising:
The stylet tail portion of the flexible stylet extends from the rear end of the flexible delivery tube, and the stylet tail portion is equipped with a stop step. The stop step is in contact with one end of the flexible delivery tube, serving as a positioning function to prevent the front end of the flexible stylet from protruding out of the trocar needle and piercing into the target body.

4. A radioactive source delivery system with a stylet pulling mechanism according to claim 1, before the radioactive source delivery, the stylet pulling component is docked with the stylet tail portion inside each flexible delivery tube, and to pull out the flexible stylet, removing the flexible stylet from the flexible delivery tube, thereby forming a hollow flexible delivery channel. The flexible stylet is a a flexible wire with elasticity, which can be bent under the external force and can be restored to a straight state after the external force is removed. The material of the flexible stylet is one or a combination of several types, including nickel-titanium alloy, spring steel, and composite materials. The length of the flexible stylet is greater than 600 mm.

5. A radioactive source delivery system with a stylet pulling mechanism according to claim 1, wherein the pushing wire is a flexible pushing wire, and the wire driving mechanism is a flexible wire driving mechanism. The flexible pushing wire is a flexible wire with elasticity, which can be bent under the external force and can be restored to a straight state after the external force is removed. The material of the flexible pushing wire is one or a combination of several types, including nickel-titanium alloy, spring steel, and composite materials. The length of the flexible pushing wire is greater than 600 mm.

6. A radioactive source delivery system with a stylet pulling mechanism according to claim 1, wherein the wire output channel docks to one of the flexible delivery tubes through a channel-switching mechanism. The front end of the flexible delivery tube is connected with a trocar needle or has a quick connector designed to connect with a trocar needle. The quick connector is fixedly connected to the trocar needle using one or a combination of thread, latch, and adhesive. The wire driving mechanism drives the pushing wire forward to push the radioactive source along the wire output channel, through the flexible delivery tube, and into the target body via the trocar needle that has been inserted into the target body.

16. 7. A radioactive source delivery system with a stylet pulling mechanism according to claim 6, wherein the radioactive source feeding mechanism is a cutting mechanism. In this case, the pushing wire itself is a seed strand or a seed strand casing, or the front half of the pushing wire is a seed strand or seed strand casing, and the rear half of the pushing wire is a pushing wire. The seed strand and seed strand casing can be cut by the cutting mechanism, and the target length of the seed strand or seed strand casing is severed from the front end of the pushing wire by the cutting mechanism, thereby achieving the feeding of the seed strand or seed strand casing. When the severed part is a seed strand casing, the radioactive source feeding mechanism also includes a seed embedding mechanism. This seed embedding mechanism is capable of inserting seeds and/or spacers into the seed strand casing from one end or the side, to form a complete seed strand. The cutting mechanism is positioned at any location along the wire output channel.
Alternatively, the radioactive source feeding mechanism uses a magazine for feeding. The radioactive source feeding mechanism is directly integrated into the wire output channel. Seeds, or seed strands, or seed strand casings are loaded into the storage slot or hole within the magazine. The feeding mechanism installed on the magazine is used to place seeds, or seed strands, or seed strand casings at the front end of the pushing wire for feeding. When the magazine is loaded with a seed strand casing, the radioactive source feeding mechanism also includes a seed embedding mechanism. This seed embedding mechanism is capable of inserting seeds and/or spacers into the seed strand casing from one end or the side, forming a complete seed strand.
Alternatively, the radioactive source feeding mechanism uses a seed strand driving mechanism for feeding seed strands. The radioactive source feeding mechanism comprises a seed strand driving mechanism, a seed strand output channel, and a cutting mechanism. It outputs seed strands or seed strand casings by the seed strand driving mechanism and cuts them to the desired length by the cutting mechanism, to achieve the feeding of seed strands or seed strand casings. When the seed strand driving mechanism outputs a seed strand casing, the radioactive source feeding mechanism also includes a seed embedding mechanism. This seed embedding mechanism is capable of inserting seeds and/or spacers into the seed strand casing from one end or the side, thereby forming a complete seed strand. The seed strand driving mechanism is connected to the seed strand output channel. The seed strand output channel is a rigid structure or a flexible structure that can be bent. When the seed strand output channel is connected with the Y-shaped channel or the second motion platform, the severed seed strand is transported to the output channel and is located ahead of the pushing wire.

8. A radioactive source delivery system with a stylet pulling mechanism according to claim 6, wherein the channel-switching mechanism includes
a first motion platform with one end of the wire output channel and the first connecting portion respectively installed on the opposite sides; and
a first connecting portion.
The first motion platform is one of the following modes:
A. The first connecting portion moves while one end of the wire output channel remains stationary;
B. The first connecting portion remains stationary while one end of the wire output channel moves;
C. The first connecting portion moves while one end of the wire output channel also moves.
The first motion platform includes a planar motion mechanism and a first front-and-back docking mechanism. The first front-and-back docking mechanism connects to the planar motion mechanism. The planar motion mechanism is used to drive the first front-and-back docking mechanism to move within a plane. The first front-and-back docking mechanism drives one end of the wire output channel or the first connecting portion to move back and forth in a direction perpendicular to that plane.
The planar motion mechanism takes two rotational movements around two axes to achieve the motion of the first front-and-back docking mechanism in a plane. Alternatively, a rotary motion in one direction and a linear motion in one direction of the planar motion mechanism drives the motion of the first front-and-back docking mechanism in a plane. Alternatively, the planar motion mechanism takes two linear motions in two directions to achieve two degrees of freedom motion of the first front-and-back docking mechanism in space.

9. A radioactive source delivery system with a stylet pulling mechanism according to claim 8, wherein the first connecting portion connects to the first connection part which has a plurality of connection holes. The one end of each flexible delivery tube is installed at the corresponding connection hole. The first motion platform is used to achieve the docking between one end of the wire output channel with each flexible delivery tube on the first connection part, forming a delivery channel for radioactive source, achieving multi-channel delivery. The first connecting portion is one or a combination of an adhesive connection portion, a welding connection portion, a threaded connection portion, a snap-fit connection portion, or a locking connection portion.

10. A radioactive source delivery system with a stylet pulling mechanism according to claim 8 further comprising:
a second motion platform with a stylet pulling mechanism and a second connection part respectively installed on different sides, which is used to achieve the relative motion in space of the stylet pulling component and the second connection part. It makes the stylet pulling component docks with the stylet tail portion of the flexible stylet inside each flexible delivery tube attached to the second connection part, performing the pulling of the flexible stylet, pulling it out from the flexible delivery tube, thereby achieving multi-channel stylet pulling. (The stylet tail portion protrudes from the connection hole of the second connection part.)
The second motion platform is one of the following modes:
A. The second connection part moves while the stylet pulling component remains stationary;
B. The second connection part remains stationary while the stylet pulling component moves;
C. The second connection part moves while the stylet pulling component also moves.
Alternatively, the second motion platform is the first motion platform. In this scenario, the stylet pulling component and one end of the wire output channel are installed at the same side of the first motion platform. The second connection part is the first connection part. The first motion platform also includes a second front-and-back docking mechanism which connects to the planar motion mechanism. The first front-and-back docking mechanism and the second front-and-back docking mechanism are respectively used for the back and forth docking motion of one end of the wire output channel with the stylet pulling component. First, the stylet pulling component docks with the stylet tail portion of the flexible stylet inside one of the flexible delivery tubes on the first connection part, and pulls the flexible stylet. After the flexible stylet pulling is completed, a new implantation channel is established. The first motion platform drives the wire output channel connects with the flexible delivery tube, and then the implantation is carried out through the newly established implantation channel.

11. A radioactive source delivery system with a stylet pulling mechanism according to claim 1 further comprising:
a stylet storage mechanism being used to store the flexible stylets pulled out by the stylet pulling component and located at the rear end of the stylet pulling component. When the flexible stylet is output from the rear end of the stylet pulling component, the stylet storage mechanism stores the flexible stylet accordingly.
The stylet storage mechanism is a wheeled storage or a stylet storage tube.

12. A radioactive source delivery system with a stylet pulling mechanism according to claim 11, wherein the wheeled storage uses a coil wheel component. The coil wheel component includes a storage wheel and a storage wheel driver. The storage wheel driver drives the storage wheel to rotate, causing the flexible stylet to be wound around the outer surface of the storage wheel.
Alternatively, the wheeled storage adopts a wheel-shaped stylet container which has a concave inner surface and has an opening on the side. The flexible stylet extends into the concave inner surface through the side opening. The wheel-shaped stylet container is designed to rotate freely or be actively driven, positioned behind the stylet pulling mechanism. Under the action of the flexible stylet's own elasticity, the flexible stylet is automatically wound within the concave inner surface of the wheel-shaped stylet container.

13. A radioactive source delivery system with a stylet pulling mechanism according to claim 11, wherein the stylet storage tube can be any one of a straight tube, a spiral tube, or a thin-film tube. The material of the stylet storage tube is one or a combination of metal, plastic, rubber, latex, silicone, or elastomer material. The inlet end of the storage tube has an elastic extension section, which can shorten under the action of press and automatically extend to its original position after the press is released.

14. A radioactive source delivery system with a stylet pulling mechanism according to claim 8 further comprising:
a stylet insertion mechanism that can insert a stylet along the flexible delivery tube into the trocar needle, filling the space inside the trocar needle, which prevents blood from flowing into the trocar needle, otherwise blood coagulation can lead to the blockage of the trocar needle.

15. A radioactive source delivery system with a stylet pulling mechanism according to claim 14, wherein the stylet pulling component is also the stylet insertion mechanism, which adopts a friction stylet pulling component. A part of this component presses tightly against the flexible stylet. The friction stylet pulling component is capable of being driven to rotate in both directions, and it uses the frictional force generated by pressing to achieve the extraction and insertion of the flexible stylet.

16. A radioactive source delivery system with a stylet pulling mechanism according to claim 15, wherein the stylet tail portion is equipped with a stop step. The stylet insertion mechanism achieves precise positioning of the flexible stylet through limiting action of the stop step and the rear end face of the flexible delivery tube. This ensures that the front end of the flexible stylet reaches the front end of the trocar needle without protruding further forward.
Alternatively, the stylet insertion mechanism is equipped with a displacement measurement device. This device can measure the actual displacement of the flexible stylet. When the displacement measurement device detects that the front end of the flexible stylet reaches the front end of the trocar needle, the stylet insertion mechanism ceases its operation.

17. A radioactive source delivery system with a stylet pulling mechanism according to claim 15 further comprising:
a third motion platform with the stylet insertion mechanism and a third connection part respectively installed on the opposite sides, which is used to achieve the relative motion in space of the stylet insertion mechanism and the third connection part. It makes the stylet insertion mechanism docks with each flexible delivery tube on the third connection part and insert the flexible stylet through that flexible delivery tube into the trocar needle, achieving multi-channel stylet insertion.
Alternatively, the third motion platform makes the stylet insertion mechanism docks with the stylet disposal tube mounted on the third connection part and insert the pulled flexible stylet into the stylet disposal tube.
The third motion platform is one of the following modes:
A. The third connection part moves while the stylet insertion mechanism remains stationary;
B. The third connection part remains stationary while the stylet insertion mechanism moves;
C. The third connection part moves while the stylet insertion mechanism also moves.
Alternatively, the third motion platform is the first motion platform. In this scenario, the stylet insertion mechanism and one end of the wire output channel are installed at the same side of the first motion platform. The third connection part is the first connection part. The first motion platform also includes a third front-and-back docking mechanism which connects to the planar motion mechanism. The third front-and-back docking mechanism and the first front-and-back docking mechanism are respectively used for the back and forth docking motion of one end of the wire output channel with the stylet insertion mechanism. First, docking the wire output channel with one of the flexible delivery tubes on the first connection part, and then proceeding with the implantation. After the implantation is completed, the stylet insertion mechanism docks with this flexible delivery tube and advances the flexible stylet through the flexible delivery tube into the trocar needle.

18. The use method of a radioactive source delivery system with a stylet pulling mechanism, wherein including the following steps:
a. Connecting the front ends of several flexible delivery tubes to the tail end of a trocar needle that has already been inserted into the target body. The flexible stylet is inside the flexible delivery tube, the front end of the flexible stylet extends to the front end of the trocar needle, so as to filling the space inside the trocar needle. A small section of the flexible stylet extends out as a tail from the rear end of the flexible delivery tube.
b. Before implanting a radioactive source through one of the trocar needles, it is necessary to dock the stylet pulling component with the flexible delivery tube connected to this trocar needle. The flexible stylet pulling component clamps to the flexible stylet tail portion which extends from the tail end of the flexible delivery tube, so as to pull out the flexible stylet from the flexible delivery tube and form a hollow delivery channel.
c. Docking one end of the wire output channel with this flexible delivery tube. The radioactive source delivery mechanism then pushes the radioactive source forward along the wire output channel, the flexible delivery tube, and the trocar needle, until it is implanted within the target body.

19. The use method of a radioactive source delivery system with a stylet pulling mechanism according to claim 18, wherein step c is implemented by the docking motion of the first motion platform. Initially, one end of the flexible delivery tube is mounted on the first connection part. One end of the wire output channel and the first connection part are respectively installed at the different ends of the first motion platform. The first motion platform is used to realize the relative motion in space between the wire output channel and the first connection part. This allows the wire output channel connects to each flexible delivery tube on the first connection part, forming a delivery channel for radioactive source, so that the multi-channel implantation is achieved. The first motion platform is one of the following modes:
A. The first connection part moves while the wire output channel remains stationary;
B. The first connection part remains stationary while the wire output channel moves;
C. The first connection part moves while the wire output channel also moves.

20. The use method of a radioactive source delivery system with a stylet pulling mechanism according to claim 18, wherein step b is implemented by the docking motion of the first motion platform. Initially, one end of a plurality of flexible delivery tubes are mounted on the first connection part. The stylet pulling component and the first connection part are respectively installed at the opposite sides of the first motion platform. By the first motion platform, relative motion in space is achieved between the stylet pulling component and the first connection part. This enables the stylet pulling component to dock with the stylet tail portion inside each flexible delivery tube on the first connection part, and to pull out the flexible stylet, then the flexible stylet is pulled out from the flexible delivery tube, accomplishing multi-channel stylet pulling out. The first motion platform is one of the following modes:
A. The first connection part moves while the the stylet pulling component remains stationary;
B. The first connection part remains stationary while the the stylet pulling component moves;
C. The first connection part moves while the the stylet pulling component also moves.
